(19) United States
(12) Patent Application Publication
(10) Pub. No.: US 2011/0212448 A1
(43) Pub. Date: Sep. 1, 2011

(54) GAMMACARBOXYGLUTAMATE-RICH PROTEIN, METHODS AND ASSAYS FOR ITS DETECTION, PURIFICATION AND QUANTIFICATION AND USES THEREOF

(76) Inventors: Dina Cristina Fernandes Rodrigues Da Costa Simes, Quarteira (PT); Carla Alexandra Sâo Bento Viegas, Faro (PT); Maria Leonor Quintais Cancela Da Fonseca, Faro (PT)

(21) Appl. No.: 13/061,317

(22) PCT Filed: Aug. 27, 2009
(86) PCT No.: PCT/PT2009/000046

§ 371 Date: May 19, 2011

Related U.S. Application Data
(60) Provisional application No. 61/136,315, filed on Aug. 27, 2008.

(51) Int. Cl. G01N 33/566; C12P 21/00; C12N 5/18; G01N 21/64; G01N 21/76; C12Q 1/68

(52) U.S. Cl. 435/6.11; 435/69.1; 435/331; 436/501; 435/7.92; 435/7.9; 435/7.1

(57) ABSTRACT

The presently disclosed subject matter refers to a gammacarboxyglutamate-rich protein that shows in vivo a high capacity to bind calcium through specific gamma carboxylated glutamic acid residues (Gla). It includes a description of the referred protein, purification procedures, protein detection and quantification tools and methods. A kit for the detection and quantification of said protein in samples is contemplated. This kit can include the use of one or more antibodies produced against the homologous sequence of the target species to be analyzed, and thus, methods for the production of such antibodies are disclosed as well. In another aspect, the methods and tools described hereby can be used as biomarkers for evaluation of presence or risk to develop certain diseases. In another aspect of the disclosed subject matter, available complete GRP cDNA and gene sequences obtained from several species also enable the in vitro production of antigens, the quantification of GRP expression, the screening of GRP polymorphisms to access the predisposition for certain diseases and the screening for GRP mutations.

### FIELD OF THE INVENTION

The present invention refers to a protein useful in biochemistry, molecular biology and molecular analysis. In particular, the invention relates to modulators of tissue calcification, particularly to a vitamin K dependent protein (VKD) a gammacarboxyglutamate rich protein (GRP), which has the capacity for binding calcium through the presence of a high number of gammacarboxyglutamate (Gla) residues.

### BACKGROUND OF THE INVENTION

Vitamin K is a cofactor in the posttranslational conversion of glutamate residues into γ-carboxyglutamate (Gla). The distribution of γ-glutamyl carboxylase expression in mammalian tissues is ubiquitous and it has been demonstrated that there is also enzyme activity in extra-hepatic tissues like bone, cartilage, vascular system and skin (Vermeer et al 1986). Naturally occurring carboxylase mutations have been informative for function and are associated with bleeding complications and, more recently, with pseudoxanthoma elasticum (PXE)-like phenotype (Vanakker, O. M. et al, 2007). Deletion of gammacarboxylase results in embryonic lethality in mice (Zhu et al 2007).

The Vitamin K-dependent protein family also includes the coagulation factors synthesized in the liver, which were the first VKD proteins discovered (prothrombin, factor VII, IX and X, protein C, S and Z, that contain between 9-13 Gla residues) (Shearer, M. J., 1990; Vermeer, C., 1990).

The presence of Gla residues in extra-hepatic tissues, namely bone and cartilage, has lead to the discovery of a new group of VKD proteins not related to haemostasis: osteocalcin (BGP) (Hauschka, P. V. et al, 1975; Price, P. A., et al., 1976) and Matrix Gla protein (MGP) (Price, P. A., et al., 1983). Both proteins contain 3 to 5 Gla residues and their function is related to the control of tissue mineralization (Laizé, V. et al., 2005). Later, additional VKD proteins were identified (gas6 (Manfioletti, G., et al., 1993), PRGP1 e PRGP2 (Kulman, J. D. et al, 1997, 2001) and found to be involved in diverse biological functions such as growth control, apoptosis and signal transduction. In all cases where their function was known, the activity of the various Gla-proteins was strictly dependent on the presence of the Gla-residues (Shearer, M. J., 1990; Vermeer, C., 1990).

The mineralization processes of calcified tissues (bone, cartilage, dentin, enamel and cement) have been largely investigated and osteocalcin is considered to be a marker of osteoblastic activity present in bony tissues and teeth and also a mineralization modulator in these tissues. Matrix Gla-protein (MGP) is the most efficient physiological inhibitor of tissue calcification presently known.

Soft tissue ectopic calcification is an abnormal mineralization process relevant for human health, namely the vascular calcification that takes place in the heart, artery walls, valves and blood vessels, and MGP has always been referred as associated to these pathologies. This correlation was originally based on the phenotype of transgenic mice deficient for MGP, which develop severe arterial calcifications after birth and die within 6-8 weeks after birth due to rupture of the aorta or one of the other main arteries (Luo, G. et al., 1997).

Humans lacking functional matrix Gla protein show calcification of cartilage and also exhibit the same type of vascular calcification observed in mice (Munroe P. B., et al 1999, Hur, D. J. et al., 2005).

Therefore, MGP-related antigens (tissue-associated and circulating) have been used as biomarkers for several diseases, namely atherosclerosis, vascular disease, rheumatic arthritis, and angiogenesis (PCT/EP00/06173) (Cranenburg E. C. M et al, 2008).

Calcifications of soft tissues are in fact a common feature in many diseases and have been reported over the years to be associated to almost all organs and tissues of the human body, either as a direct cause of the disease or as a secondary effect of the pathology.

Human skin, for example, is an organ containing the complete biochemical machinery to develop calcification, as shown by the high abundance of reported pathologies ranging from mild to very severe skin calcifications such as dermatomyositis, scleroderma, pseudoxanthoma elasticum (PXE) calciphylaxis and Keutel syndrome (Borst P et al, 2008, Davies, C. A. et al, 2006, Oliveri M B et al, 1996; Lobo I M, et al., 2008; Van Summeren M J et al, 2008; Li Q et al, 2007 and references therein).

In fact, more than 20 years ago, Lian et al. referred the presence of Gla containing proteins associated with skin calcifications and subcutaneous plaques in patients with juvenile dermatomyositis and scleroderma with calcinosis (Lian, J B, et al., 1982). Nonetheless, this researcher only refers to the presence of non-identified Gla-containing proteins associated with the subcutaneous calcifications, suggesting the involvement of these proteins in soft tissues pathological calcifications (Lian, J B, et al., 1982). Later, this researcher established the origin of this Gla protein, in calcergy and calciphylaxis patients skin, by positive identification of osteocalcin by radioimmunoassay in the apatite-like crystals (Lian J B, et al 1983).

A few years later, de Boer-van den Berg M A, (1986) corroborated this hypothesis when they identified the presence of a high gammacarboxylase activity in mouse epidermis and dermis and also strongly suggested the presence of other non-identified gla-containing protein(s) associated with dermal calcifications pathologies namely in scleroderma or dermatomyositis pathologies.

Later, this researcher was able to establish a direct association between the presence of MGP in its several possible forms of processing (gammacarboxylation/phosphorylation) with the appearance of symptoms of several skin calcification pathologies, namely pseudoxanthomas elasticum (PXE) (Li Q et al, 2007) and juvenile dermatomyositis (Summeren M J et al, 2008).

Nonetheless, the only reference published until now, that relates the ectopic calcifications in skin or in other tissues to a VKD protein as been limited to MGP and osteocalcin (Li Q et al, 2007, Summeren M J et al, 2008, Davies, C. A. et al, 2006, Lian et al, 1983).

The extracellular mineralization has been referred as a passive process while its inhibition is thought to be an active process since mineralization does appear when calcification inhibitors are not present. In fact, there are several other proteins beside MGP that have been proven to have a function as mineralization inhibitors like osteocalcin (BGP), bone sialoprotein (BSP), osteoprotegerin (Opg) and fetuin. Nonetheless, the work already published referring the development of transgenic knock out mice lacking each of these proteins, with the exception of MGP, have not reported any skin or hair follicle abnormalities or calcifications.

On the other hand, there are several reported cases of regression of recent subcutaneous calcifications in patients with scleroderma and dermatomyositis following treatment with low doses of warfarin (Cukierman, T. et al., 2004; Matsuoka, Y. et al, 1998). In fact, this compound affects functional expression of several Gla-containing proteins, namely MGP and BGP in vitro (Barone, L. M. et al, 1994) and also induces the accumulation of non-gammacarboxylated VKD-proteins at microssome levels, both in dermis and epidermis (de Boer-van den Berg M A, 1986). It has also been proposed that skin necrosis is related to the function of a skin Gla protein that is regulated by skin carboxylase activity (Green D., 1984, de Boer-van den Berg M A, 1986).

Recently, two papers were published identifying a mouse cDNA that appears to correspond to the protein sequence presented in this invention (Gla-rich protein, GRP). Nonetheless, these authors refer to the protein as a unique cartilage associated protein in mouse (UCMA-Unique Cartilage-Associated Protein, recently renamed Upper zone of growth plate and cartilage matrix associated, and they propose that it may function as a chondrogenenic marker in mammals, based on their results that show protein expression only in cartilaginous cells and accumulation of both processed and unprocessed forms of UCMA in the cartilaginous extracellular matrix. However, the only UCMA processed form reported by these authors is the propeptide processing made by a furin-like enzyme (Tagariello, A., et al., 2008 and Surmann-Schmitt, C., et al., 2008).

In contrast to their description, the results showed in the present invention, performed in rat and human, confirm that GRP protein is present in several different types of biological fluids, tissues and cells therein and for this reason it is not cartilage-specific and so the name given by these authors is inadequate. Furthermore, their reported findings do not correspond to the actual tissue distribution of this protein in rat/humans.

More importantly, these authors (Surmann-Schmitt C, et al., 2008, Tagariello A, et al., 2008) also failed to identify the presence of Gla residues in the mature processed protein. In their report they refer the production of recombinant protein for antibody production and validation, but the methodology used could never provide a fully gammacarboxylated form and for that reason the resulting protein would never be fully functional. The presence of Gla residues in a protein, responsible for greatly enhancing its calcium binding capacity, is crucial for its physiological function and of major relevance for this invention as well as for its use as an analytic tool, as well as for in vivo and in vitro studies.

These authors (Surmann-Schmitt C, et al., 2008, Tagariello A, et al., 2008) also failed to identify several other protein functional motifs that contribute to its classification as a vitamin K-dependent (VKD) protein. In addition, in their report the authors were restricted to the use of cartilaginous tissues so it was impossible for them to see that in fact the GRP is much more widely expressed in different tissues in the organism and also accumulates in many different mammalian/human tissues including skin, cartilage, bone, vascular system, nervous system, among others.

At the cell type level, the results presented by the authors Surmann-Schmitt C, et al., 2008, Tagariello A, et al., 2008, concerning the sites of protein expression, even when taken into consideration the fact that they were limited to the mouse (Mus musculus) model, were also insufficient because they only report detection of protein expression in cartilaginous cells, a result that does not reflect the full tissue distribution of this protein in rodents.

In conclusion, the previous two articles by these authors do not show nor suggest a relationship between UCMA or a Gla-rich protein (GRP) and non-cartilaginous tissues as, for example, vascular system or skin, whether at the expression or at the accumulation levels.

Furthermore, it is also a fact that there is no report nor suggestion for the use of such protein (GRP) as a marker for diseases related to abnormal calcifications, or the like, nor does it disclose an assay for it either for quantification or detection of the protein or its mRNA in any source (for example biopsies or biological fluids) for the purpose of its use in analytical methods.

### GENERAL DESCRIPTION OF THE INVENTION

This invention refers to a new member of the VKD protein family containing an unprecedented high density of Gla residues (22%) and thus it refers to a Gla-rich protein (GRP).

This protein was initially isolated and purified from sturgeon (Acipenser nacarii) calcified cartilage using a well described method for the extraction of mineral binding proteins as for example osteocalcin and matrix gla protein (MGP) (Simes D. C. et al, 2003, 2004). This methodology allows the selective extraction and purification of proteins that are originally bound to the tissue-calcified matrix (FIG. 1).

The presence of this protein in other organisms, namely in mammals, was further performed and, surprisingly, it was also detected (orthologs) in all vertebrate taxonomic groups including human.

The isolation and purification of the mature GRP extracted from mineralized tissue allowed the identification of the functional motifs (Gla residues) that are responsible for its high calcium binding ability (Table 1).

TABLE 1
Species Acronym

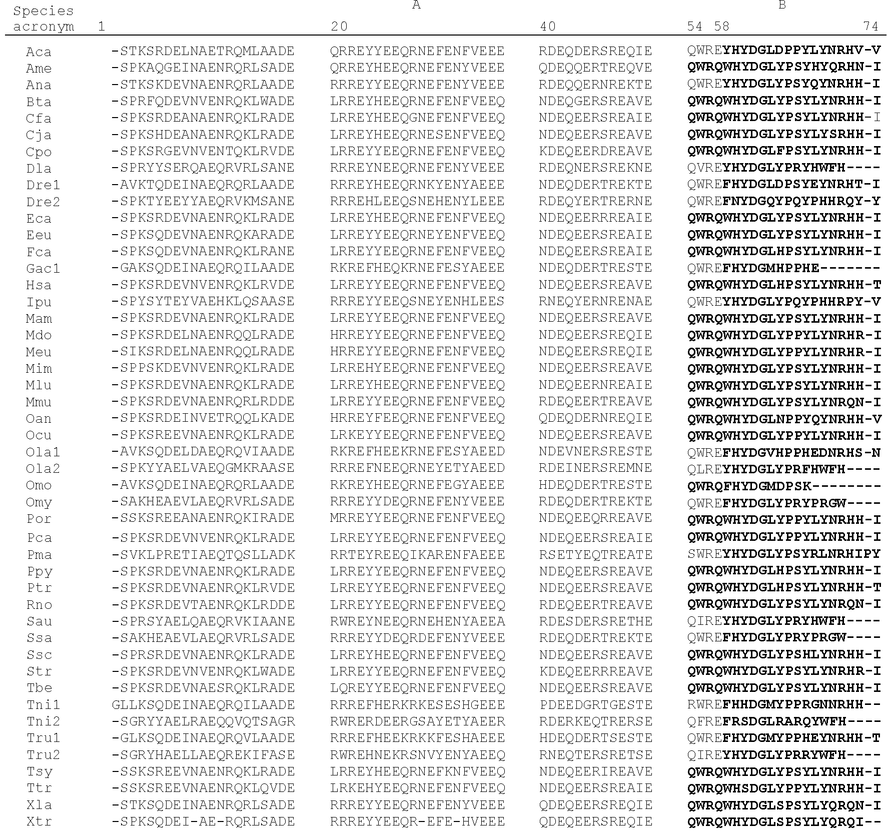

This would not be possible based on the simple observation of the deduced cDNA sequence since the functional motifs results from a post-translation modification catalyzed by the gamma-carboxylase.

Therefore, although the cDNA sequence is available in the databases, the correspondent functional protein has never been reported.

This small protein (10.2 kDa) contains the highest ratio of Gla residues/size ever found in any other known VKD protein (14-16 Gla residues in a total of 74-65 amino acids depending on the species, Table 1), conferring it an outstanding capacity for interaction with calcium.

The presence of these Gla residues in GRP was confirmed using a specific SDS-PAGE Gla-containing proteins staining method (DBS staining method; Simes D. C. et al, 2003, 2004) (FIG. 1). The number of Gla residues in GRP was further quantified by amino acid analysis of the purified protein (FIG. 1) using both acid and alkaline hydrolysis conditions (Table 2).

Based on these results, it was possible to confirm the existence of 100% gammacarboxylation in all 16 Glu residues (Table 2) in sturgeon GRP and also the inexistence of any other post-translation modification, as for example, phosphorylation and/or glycosylation.

The remarkably high degree of conservation among orthologs of GRP—identified in 48 different vertebrate species from most classes within these phyla and absent in invertebrates, allowed us to confirm that the protein has a function highly conserved throughout vertebrate evolution illustrated by the fact that there are—68 identical residues between sturgeon and human GRP including its propeptide and the mature form (Table 1).

TABLE 2
Amino acid analyses of sturgeon GRP.
Ten and 30 μg of the G-75 purified GRP were subjected to
acid and alkaline hydrolysis respectively in order to
determine the amino acid composition of GRP and quantify
the amount of Gla residues in the protein.

| | | | Alkaline | |
|---|---|---|---|---|
| | Acid hydrolysis | | hydrolysis | |
| Amino acid | Found | Predicted | Found | Predicted |
| γ-Carboxyglutamic | | | 16.7 | 16* |
| acid | | | | |
| Aspartic acid | 10.3 | 10 | 8.6 | 10 |
| Threonine | 2.2 | 2 | | |
| Serine | 3.2 | 3 | | |
| Glutamic acid | 19.9 | 22** | 5.3 | 6 |
| Proline | 1.7 | 1 | | |
| Glycine | 2.5 | 1 | | |
| Alanine | 4.1 | 3 | | |
| Valine | 2.0 | 2 | | |
| Cysteine | 0 | 0 | | |
| Methionine | 0 | 0 | | |
| Isoleucine | 0.8 | 1 | | |
| Leucine | 2.4 | 2 | | |
| Tyrosine | 6.3 | 8 | | |
| Phenylalanine | 1.3 | 1 | | |
| Lysine | 3.1 | 3 | | |
| Histidine | 2.9 | 3 | | |
| Arginine | 10.3 | 11 | | |
| Tryptophan | N.D. | 1 | | |

*Prediction assumes γ-carboxylation of all 16 Glu residues in GRP.
**16 Glu + 6 Gln;
N.D., not detected.

When comparing the GRP protein with all other known VKD proteins, it is observed that the specific motifs essential for their function are all present and well conserved (Table 1) namely:
(1) a signal peptide including 26-27 aa;
(2) a propeptide domain containing a gamma-carboxylase recognition site, an AXXF motif and a furin-like cleavage site;
(3) a proven Gla-containing mature protein with 65-74 amino acids containing in GRP 14-16 Gla residues, depending on the species.

However, the most striking features that distinguish this protein from other VKD protein known to date and that give it its uniqueness are:
(1) the GRP Gla motif has no homology with the Gla motifs of other known VKD proteins;
(2) the GRP Gla motif spreads around the complete mature protein and contains 14-16 Gla residues (depending on the species);
(3) the relative position of Gla residues is highly conserved from sturgeon to man.

The recent identification of a new vitamin K dependent protein (i.e. GRP) containing Gla residues for calcium binding, as well as the localization at the cell level, of both the expression (FIGS. 2 and 3) and the accumulation (FIG. 4) pattern of this protein, represents a relevant advance for the understanding of the mechanisms involving ectopic calcification and related pathologies.

The results, included in the present invention, referring to rat, pig and human and also disclosed by FIG. 9 show that the GRP protein pattern of accumulation is very wide since it is present in blood serum and plasma (FIG. 8), in skin (dermis and epidermis) and its appendages (hair follicles, sebaceous and sweat glands) (FIGS. 3.2 and 4.2), in vascular and nervous system (FIGS. 3.3 and 4.3), in bone (FIGS. 3.1 and 4.1), in all types of cartilage (FIGS. 3.1, 3.2, 4.1 and 4.2), in muscle fibers, in brain, in heart, in eye, in hemopoietic tissue, and in all irrigated organs. Many cells types that are part of this tissues and organs were shown to express GRP namely for example osteoblasts, fibroblasts, vascular smooth muscle cells, chondrocytes, chondroblasts, dermatocytes, keratinocytes, neurons, glial cells, leucocytes, cardiac and Purkinje fibers.

These results are highly relevant since it suggests a function in global calcium modulation in vertebrates and relates this protein to several pathologies involving, or associated with, calcifications that can appear at multiple locations of the organism.

The GRP Gla residues are essential for the function of the protein as calcium modulator in all tissues. In fact, the presence of this Gla motif gives the protein the ability to bind calcium, either in the mineral or salt form (hydroxyapatite crystals or amorphous calcium phosphates) or in the soluble form, through a chemical bridge involving negatively charged molecules present in the extracellular matrix and connective tissues of all the tissues analyzed, as for example glycosaminoglycans (hyaluronic acid, dermatan sulphate, chondroitin sulfate queratan sulphate, or heparan sulphate depending on the specific tissue).

Based on the high calcium affinity of the protein together with our in situ and immunohistochemical results, both in rat and human, the methods and tools herein disclosed are very useful (alone or together with others) as evaluation and analysis tools for certain diseases, for example, atherosclerosis and other vascular diseases, scleroderma with or without calcinosis, pseudoxanthoma elasticum, dermatomyositis with or without calcinosis and other skin calcification pathologies, bone and cartilage related pathologies osteoporosis and metabolic disorders leading to abnormal calcification occurring in either soft or mineralized tissues, as diabetes, among others.

As an example, the results included in the present invention describe the GRP immunolocalization in human samples (FIG. 6), derived from patients diagnosed with skin and vascular system-associated calcification pathologies (FIG. 7). The presence of mineral deposits in samples from patients diagnosed with dermatomyositis with calcinosis and pseudoxanthoma elasticum (PXE) were identified by von Kossa staining and the presence of GRP, accumulated at the same sites, was detected by immunohistochemistry. GRP was found to be highly accumulated at sites of calcification in both pathological situations, either when massive calcified material was deposited in the reticular dermis, or when small-calcified spots were diffused along the elastic fibers (FIG. 7). Results clearly show that GRP is associated with the mineralized material since mineral staining and GRP accumulation are perfectly co-localized.

Arterial calcification is a very common process that normally progresses with age, and in fact, up to 95% of men and women at autopsy show coronary artery calcification regardless of death cause. Also, among the normal population, patients with chronic kidney disease (CDK) are a high risk group for development of vascular calcifications. The pattern of GRP accumulation was thus studied in a group of patients with CDK and in a group of samples collected at autopsy from vascular tissue showing ectopic calcifications, as detected by von Kossa staining. In both groups the calcification observed by von Kossa staining was at the media level, characterized by the absence of macrophages and lipids. Accordingly, GRP was found to be nearly absent from the extracellular matrix detected in normal arterial wall tissues and highly accumulated at sites of medial calcification, either in mildly calcified arteries, showing disperse mineral deposits, or in extensive and advanced lesions.

In cases where calcification is homogenously dispersed along the fibers of the tunica media, GRP is detected both inside the vascular smooth muscle cells and in the extracellular matrix, in contrast with it's almost absence in non-calcified areas. When calcification is localized, either as disperse or massive mineral deposits, GRP can be easily identified as being co-localized with the mineral.

Based in these results it is surprisingly evident that the protein is associated with soft tissue calcification pathologies and ectopic calcifications.

Other ectopic calcifications may also be considered as being of considerable importance in the scope of the present invention, especially those that are associated with trauma, repetitive stress, surgery, and/or biological or non-biological implants.

Also we could conclude, based on immune-based techniques results (like immunohistochemistry (FIG. 5) and western blot and dot blot (FIG. 9), that the GRP accumulated in skin and in vascular tissues is gammacarboxylated.

The complete amino acid sequence of GRP obtained for several mammalian species including human, bovine, rat, mouse, among others, as well as for amphibians, reptiles, several teleost and cartilaginous fish, and lamprey (Table 1) led to the development of several useful methodologies and/or tools disclosed in this invention namely:

(1) Isolation and purification of GRP protein obtained from a natural source or recombinant GRP, using several sources, mammalian or non-mammalian tissues or biological fluids, and cell culture and other suitable expression systems;
(2) Production of antibodies either mono or polyclonal antibodies of class IgG, for GRP or any of its processed products, specific for different organisms depending on the specific GRP antigen to be assayed. The antibodies can be produced against the GRP protein extracted from a natural source or against a peptide analogous to its sequence (depicted in Table 1) or to different forms of its sequence, as for example different numbers of Glu residues that are gamma carboxylated, leading to different Gla contents;
(3) a diagnostic kit for detecting and quantifying GRP in samples, such as serum/plasma samples preferably from a human source but also from other non-human mammal or non-mammalian organism (using in each case a specific antibody validated for the GRP of the selected organism);
(4) an immunoassay method, for detecting and quantify the said protein in samples, such as serum or plasma sample or other source sample or extract obtained from, for example, biological fluids, cell cultures (from mammalian or non-mammalian origin), biopsies, organs, tissues or even a complete organism. The immunoassay method is based on the measurement of the extent of the antigen/specific antibody interaction and can be any one of the known immunoassay methods suitable for the purpose, such as an enzyme immunoassay, radioimmunoassay, chemiluminescence- or fluoro-immunoassay, etc. Such immunoassay systems are well known to a person skilled in the art;
(5) a process for monitoring or detecting a disease that consists in exposing a mammalian serum sample to the specific referred antibodies and determining the level of GRP in referred serum samples. This information is very useful when monitoring or detecting coronary atherosclerosis, vascular calcification angiogenesis (a disease of the vascular system), calcific uremic arteriolopathy, renal insufficiency, diabetes mellitus or ectopic calcification in skin or even in tumor development, among others;
(6) other immunoassay methods suitable for detection or quantification of GRP presence or its different processed forms, are described and can include immunohistochemistry techniques, western blot, dot blot or ELISA-based assays.

For the diagnosis of human patients, the antibody is preferably anti-human GRP. Similarly, if the organism is an non-human animal, the antibody is preferably anti- to the GRP of the same species, e.g. anti-bovine GRP if the organism is bovine. Often, there is some cross reactivity of the antibodies between species, and to confirm the specificity of the one in use, western blot analysis should always be performed each time a different antibody batch is used, using as antigens, GRPs purified from different species including the one that is assayed.

The tools provided in the scope of this invention include polyclonal antibodies obtained in rabbit or other appropriate animal, raised against either the mature purified protein extracted from tissue or a suitable biological fluid, or against a synthetic peptide homologous to specific GRP residues from adequate source, or against a synthetic peptide homologous to specific processed or unprocessed regions of GRP or against the recombinant GRP protein produced in eukaryotic cells or in other appropriated in vitro systems. In this case, one should take care to confirm that the recombinant protein is conveniently processed and gamma carboxylated, a modification which has been shown in the past to be a major problem when producing in vitro recombinant VKD proteins.

In a preferred embodiment of the invention, monoclonal antibodies of class IgG are also provided for use in described diagnostic immunoassay. These can be obtained by hybridomas formed by fusion of cells from a mouse myeloma and spleen cells from a mouse previously immunized with a peptide homologous to specific human GRP residues (selected from sequences described in Table 1), in particular one of human GRP residues 54-74, and human GRP residues 20-40, which antibodies are also referred to herein as CTermGRP and GlaGRP respectively that are preferred antibodies.

In another aspect of this invention, available complete GRP cDNA and gene sequences obtained from mammalian and non-mammalian species also enable the:

(1) Development of in vitro/in vivo expression systems for producing different forms of GRP products for therapeutic/research applications, for example the production of antigens;
(2) quantification of GRP mRNA levels in blood, tissues, biopsies, organs, cell cultures, or others biological samples, and whenever possible its correlation with the levels of accumulated protein;
(3) construction of mRNA probes for the study and evaluation of GRP expression in tissues, biopsies, organs, cell cultures, or others, and whenever possible its correlation with the levels of accumulated protein;
(4) screening for GRP mutations either in the coding sequences or in flanking/intronic regions of the corresponding genes, and its correlation with certain diseases;
(5) screening for GRP polymorphisms among different populations and different pathologies to access for predisposition and/or diagnose certain diseases;
(6) screening for GRP inherited mutations associated to predisposition and/or diagnose of certain diseases, useful for example in prenatal diagnosis of possible malformations and/or to ascertain predisposition for specific pathologies.

These and other aspects of the present invention will be more completely outlined in the detailed description that follows.

### BRIEF DESCRIPTION OF THE DRAWINGS

TABLE 1—Illustrates the GRP mature protein amino acid sequences used to design synthetic peptides to raise specific antibodies that are part of this invention. Region A is boxed with a dashed line and includes de gammacarboxylated part of the protein [contains Glu/Gla residues (E) in the sequence therein] and ranges from residue 1-54 or 58 depending on the organism or species (all in box A except the shadow light gray amino acid sequences). Region B is boxed with a continuous line and represents the part of the protein that does not contain any Glu/Gla residue and ranges from residue 54 or 58 to 74 of the protein depending on the organism (shadow in light gray).

In this Table, for each species we used an acronym. The name of the species (italic), acronym (in bold) and corresponding common names are:

Cavia porcellus Cpo Guinea pig; Mus musculus Mmu house mouse; Rattus norvegicus Rno Norway rat; Spermophilus tridecemlineatus Str thirteen-lined ground squirrel; Equus caballus Eca horse; Bos taurus Bta cattle; Sus scrofa Ssc pig; Felis catus Fca domestic cat; Canis familiaris Cfa domestic dog; Erinaceus europaeus Eeu western European hedgehog; Callithrix jacchus Cja white-tufted-ear marmoset; Macaca mulatta Mam rhesus monkey; Tarsius syrichta Tsy Philippine tarsier; Pan troglodytes Ptr chimpanzee; Pongo pygmaeus Ppy orang-utan; Homo sapiens Hsa human; Microcebus murinus Mim gray mouse lemur; Myotis lucifugus Mlu little brown bat; Oryctolagus cuniculus Ocu European rabbit; Ochotona princeps Opr American pika; Procavia capensis Pca cape rock hyrax; Tupaia belangeri Tbe northern tree shrew; Tursiops truncatus Ttr bottlenose dolphin; Macropus eugenii Meu Tammar wallaby; Monodelphis domestica Mdo gray short-tailed opossum; Ornithorhynchus anatinus Oan platypus; Anolis carolinensis Aca green anole; Ambystoma mexicanum Ame axolotl; Xenopus tropicalis Xtr western clawed frog; Xenopus laevis Xla African clawed frog; Acipenser nacarii Ana Adriatic sturgeon; Danio rerio Dre zebrafish; Ictalurus punctatus Ipu channel catfish; Oncorhynchus mykiss Omy rainbow trout; Salmo salar Ssa Atlantic salmon; Osmerus mordax; Omo rainbow smelt; Takifugu rubripes Tru torafugu; Tetraodon nigroviridis Tni spotted green pufferfish; Oryzias latipes Ola Japanese medaka; Dicentrarchus labrax Dla European seabass; Sparus aurata Sau gilthead seabream; Gasterosteus aculeatus Gac threespine stickleback; Petromyzon marinus Pma sea lamprey. The species acronym followed by number 1 or number 2 represents GRP isoform 1 and 2 respectively.

TABLE 2—Shows the 16 Gla residues of sturgeon GRP, determined by amino acid analyses. Ten and 30 μg of the G-75 purified GRP were subjected to acid and alkaline hydrolysis respectively in order to determine the amino acid composition of GRP and quantify the amount of Gla residues in the protein.

FIG. 1—Illustrates one of the possible approaches for GRP isolation and purification from calcified tissues, as described in A1 section. FIG. 1 shows the isolation (A) and purification (B and C) of Adriatic sturgeon GRP. (A) SDS-PAGE analysis of crude precipitate after acid extraction of branchial arches. Gel was stained with Coommassie Brilliant Blue (CBB) and 4-diazobenzenesulfonic acid (DBS). (B) RPLC separation of proteins in crude precipitate. Fractions of 1 ml were collected and absorbance was determined at 220 nm. (C) Purification of GRP by Sephadex G-75 chromatography. Fractions of 0.5 ml were collected and absorbance was determined at 220 nm. Four μg of RPLC fractions 49-52 and 2 μg of Sephadex G-75 fractions 16 and 28-38 were analyzed by SDS-PAGE and stained with CBB (insert in panel C).

FIG. 2—Illustrates the quantification of GRP mRNA by real-time Time PC, as an example, in a variety of tissues from adult rat (A) and sturgeon (B). (A) Levels of GRP gene expression measured by real-time qTime PC in rat adult tissues related to levels in lung (Lu). Sp, spleen; Br, brain; Ts, testis; He, heart; Ki, kidney; Pa, pancreas; Li, liver; Fe, femur; Te, teeth; Ri, rib; Sk, skull; IE, inner ear; Ta, tail; OE, outer ear; No, nose. (B) Levels of GRP gene expression measured by real-time qTime PC in adult sturgeon tissues related to levels in heart (Ht). Lv, liver; Kd, kidney; Ms, muscle; Gn, gonads; Br, brain; GP, ganoid plate; AK, anterior kidney; Sl, spleen; Sp, spine; Ct, cleithrum; HP, head plate; Op, operculum; Sk, skull; Md, mandibula; BA, branchial arches; AV, anterior vertebra; PV, posterior vertebra.

FIG. 3—Illustrates sites of GRP expression, at single cell resolution, in adult rat tissues, as determined by in situ hybridization, showing GRP mRNA in a variety of cells from mineralized and soft tissues.

FIG. 3.1—Shows GRP expressing cells in cartilaginous and bony tissues, here represented with sections of ribs (A), vertebra (B), femur (C), and tail (D). GRP is widely expressed in cartilaginous cells where it is strongly detected in immature (IC), proliferating (PC) and mature (MC) chondrocytes in the hyaline cartilage (HyC) from the ribs (6.1A 1, 1′, 2, 2′), and in columnar (CC) and hypertrophic (HC) chondrocytes in the hypertrophic zone (HZ) in ribs (6.1A 1, 1′), vertebra (6.1B 1, 1′), femur (6.1C 1, 1′) and tail (6.1C 1, 1′). GRP mRNA is also expressed in chondrocytes of fibrocartilage (IG) in vertebra (6.1B 2). In trabecular bone (TB) GRP mRNA is detected in osteoblasts (white arrow) and in osteocytes (black arrow head) in the ribs (3.1A 1, 1′), vertebra (3.1B 1, 1′), femur (6.1C 1, 1′, 2), and tail (3.1D 1, 1′). Also the bone marrow (red asterisk) in trabecular bone from ribs, vertebra and femur, is intensively stained for GRP mRNA.

FIG. 3.2—Shows GRP expression in cells from the skin and its appendages, and in the elastic cartilage, here illustrated with sections of rat ear. GRP is highly expressed either at the epidermis (Ep) (FIG. 3.2 A, C) as at dermis (De) (FIG. 3.2 A, C) levels, in the fibroblasts (b) that compose the reticular layer, and in the hair follicles (HF) (FIG. 3.2 D, E) and sebaceous glands (SG) (FIG. 3.2 F), that housing in the dermis. Besides hyaline cartilage and fibrocartilage (FIG. 3.1) GRP is also expressed in the elastic cartilage (EC) from the ears (FIG. 3.2 A, B).

FIG. 3.3—Shows GRP expression in cells from the vascular system (A) and nervous tissue (B). (A) GRP is detected in the vascular smooth muscle cells in the tunica intima (TI) of the aorta (3.3A 1), small arteries (3.3A 2), and veins (3.3A 3), but also in the tunica media (TM) of the aorta (3.3A 1), and small arteries (3.3A 2). (B) GRP is expressed in cells located in the spinal cord of rat vertebra, in particular at the level of the gray matter (GM), in the neurons (Nr), glial cells (GC), and cells of the central canal (CC). WM, white matter.

FIG. 4—Illustrates sites of GRP accumulation in adult rat tissues, as determined by immunohistochemistry using the CTermGRP antibody, showing its association with mineralized and non-mineralized matrixes, in mineralized and soft tissues.

FIG. 4.1—Shows GRP accumulation inside chondrocytes, in all stages of maturation (4.1 A, B, C, D), and in osteocytes (Oc) (7.1 F), in concordance with the ISH results (FIG. 3.1). Outside the cells GRP is accumulated in the cartilaginous non-mineralized matrix (white asterisk) (4.1 C), and in the mineralized matrix, either in the hypertrophic zone of calcified cartilage (CMM) (black asterisk) (4.1 C, B), as in bone (BMM) (black asterisk) (4.1 E).

FIG. 4.2—Shows GRP in the skin and its appendages, as well as in the elastic cartilage from the ear. In the skin GRP is highly accumulated in the epidermis (Ep) (4.2 A, B, C), as well as in the dermis (De) where it is detected in the connective tissue constituted by fibroblasts (Fb) (4.2 A, B, C), in the hair follicles (HF) (4.2 B, C), and sebaceous glands (SG) (4.2 B, C). GRP is also accumulated in the elastic cartilage (EC) (4.2 D).

FIG. 4.3—Shows GRP highly accumulated in the vascular system, in veins (Vn) (4.3 A), arteries (At) (4.3 B), and small blood capillaries (4.3 C, D). In the arteries GRP is present either in the tunica intima (TI) as in the media (TM).

FIG. 5-Illustrates the γ-carboxylation status of GRP. The M3B antibody (American Diagnostic, USA) that specifically recognizes Gla residues was used in sections of skin and ribs to immunolocalize Gla proteins. The perfect co-localization obtained with the M3B and CTermGRP antibodies (FIGS. 4.1 and 4.2), either in skin as in cartilage, shows that GRP is γ-carboxylated in these tissues. Furthermore, as far as our knowledge, there is no other Gla protein described in the literature that has this accumulation pattern in skin, e.g. in the epidermis (Ep), dermis (De), hair follicles (HF), and sebaceous glands (SG) (8A, B, C). MGP is a Gla-containing protein known to be present in cartilage and produced by chondrocytes, but so far its presence as never been associated with these particular hypertrophic chondrocytes (HC) localized within the hypertrophic mineralized matrix (back asterisk) (8D).

FIG. 6—Illustrates the GRP expression, accumulation and γ-carboxylation in healthy human skin. The patterns of GRP expression, determined by in situ hybridization (GRP ISH), and accumulation, as determined by immunolocalization using the CTermGRP antibody, are similar to the rat skin, being detected in the same structures, e.g. epidermis (Ep), dermis (De), fibroblasts of the connective tissue (Fb), hair follicles (HF) and sebaceous glands. Furthermore, GRP could be detected in the immune cells (IC) that are involved in the defense against foreign invaders passing through the epidermis. The co-localization obtained using both the CTermGRP and M3B (American Diagnostic) antibodies, shows that GRP is γ-carboxylated also in human skin.

FIG. 7—Illustrates the GRP involvement in normal skin (A) and human skin pathologies like scleroderma (B), and dermatomyositis, without calcinosis (C), and with calcinosis (D, E, F and G), in particular its association with the calcified nodules (CN) associated with calcinosis. Scleroderma and dermatomyositis are diseases characterized by the disarrangement of the connective tissue, with excessive deposits of collagen and muscle inflammation. In scleroderma samples, GRP almost withdraw from the fibroblasts of the connective tissue (10 B, B′), and is higher accumulated in the epidermis of scleroderma samples (10 B), when compared to the healthy skin (10 A). In dermatomyositis without calcinosis condition, GRP is also higher accumulated in the epidermis (10 C), when compared to the healthy skin (10 A). Remarkably, in dermatomyositis with calcinosis, GRP is highly associated and bound to the calcium deposits that form the calcified nodules (CN), either at the mineral growing front as within the mineral (10 D, E, F, and G).

FIG. 8—Illustrates the presence of GRP in human blood serum. 5 μl of total human serum (TSe), prepared as described in the A3 section, were blotted into a nitrocellulose membrane (Amersham Biosciences), as well as 5 μl of each of the fractions (F1, F2, F3) containing the low abundant proteins, resulting from the separation of the high abundant proteins present in serum, obtained with the ProteoMiner™ Kit (Bio-Rad), according to the manufacture's instructions, and described in the examples A3 section. GRP was detected using the affinity purified CTermGRP antibody.

FIG. 9—Validation of the CTerm-GRP antibody against sturgeon, rat, pig and human GRP proteins (A) and cross-reactivity of rat and sturgeon GRP isolated proteins with the Gla-specific antibody M3B (B). (A) Western blot analysis of crude rat skin 4M guanidine extract (Rat), pig skin extract of hydroxyapatite-binding proteins obtained after incubation of the 4M guanidine pig skin extract with 0.1% hydroxyapatite and further demineralization with 10% formic acid as described in the materials and methods section (Pig), and human blood vessels guanidine crude extract (Human). CTerm-GRP was used as primary antibody and alkaline phosphatase-labeled goat anti-mouse IgG as secondary antibody. Purified sturgeon GRP (Sturgeon) was used as positive control. (B) Rat GRP containing fraction, obtained by RP-HPLC (rGRP) from the skin 4M guanidine extract, was further analysed by dot blot for the presence of Gla residues with the M3B antibody and CTerm-GRP for comparison. Alkaline phosphatase-labeled goat anti-mouse IgG was used as secondary antibody and purified sturgeon GRP (StGRP) as positive control.

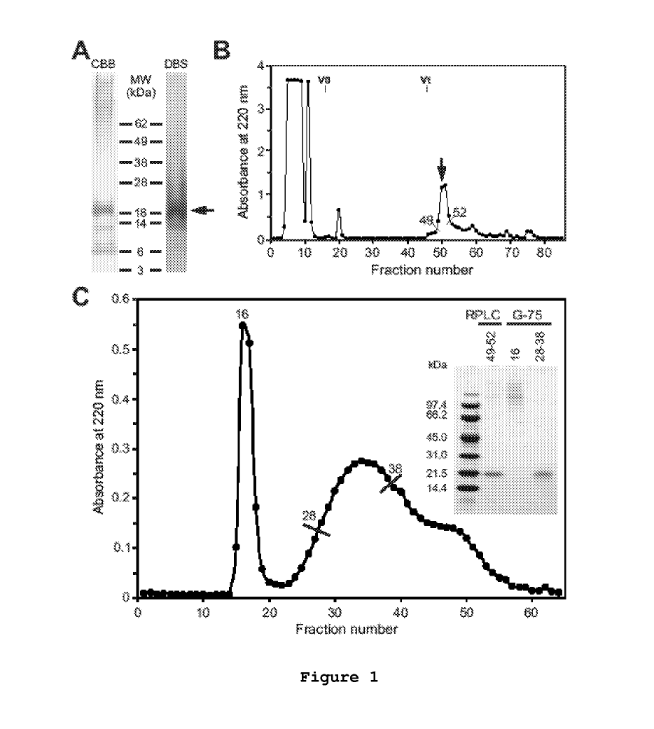

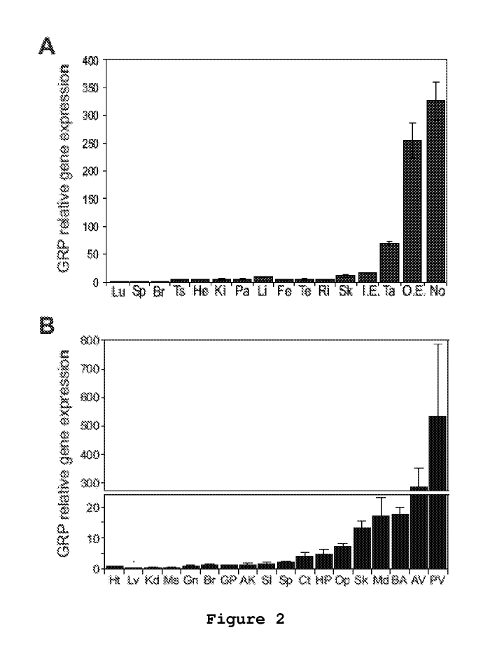

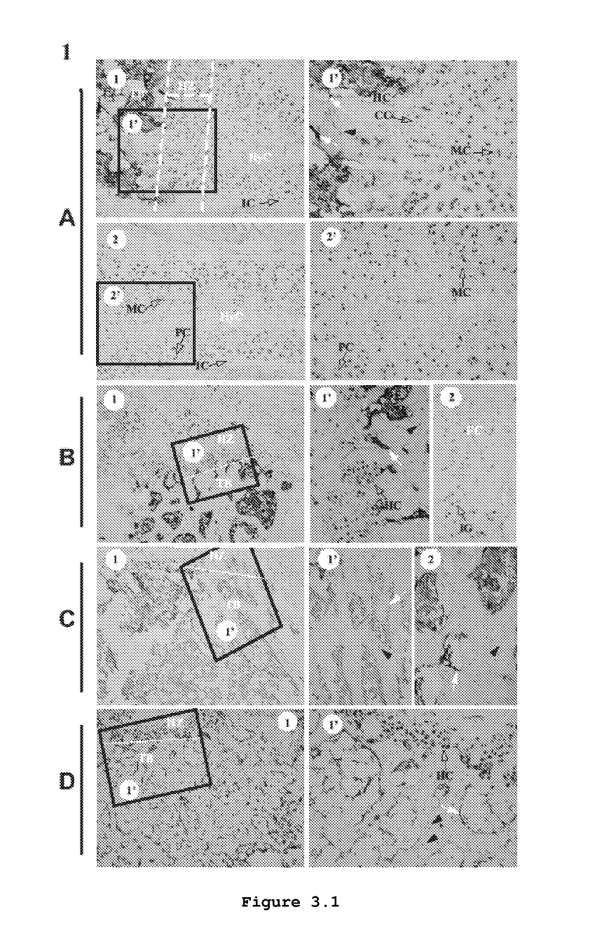

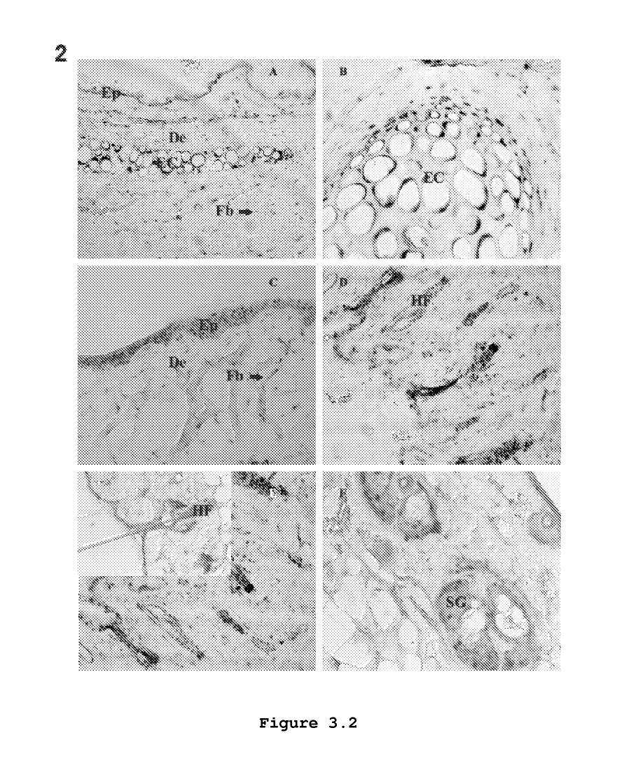

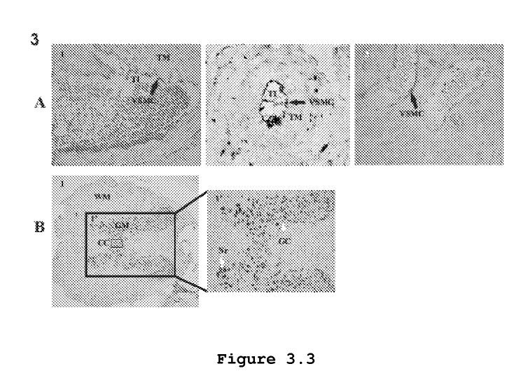

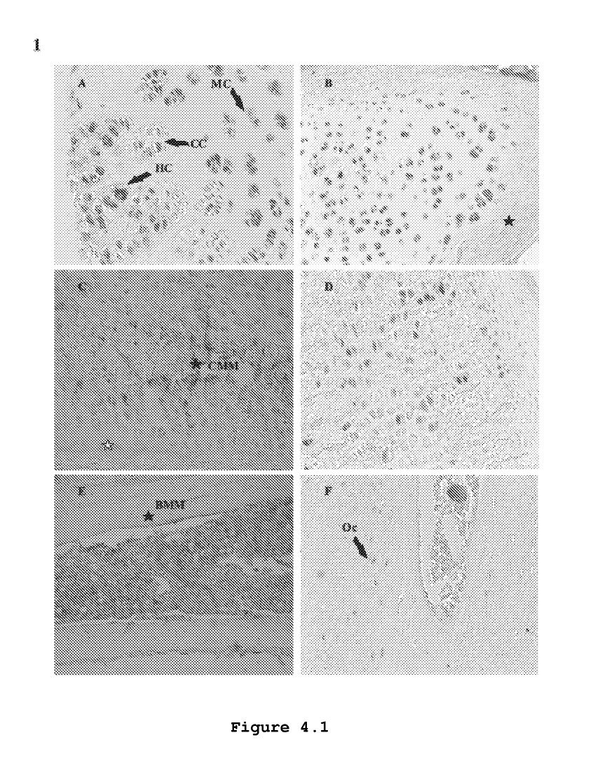

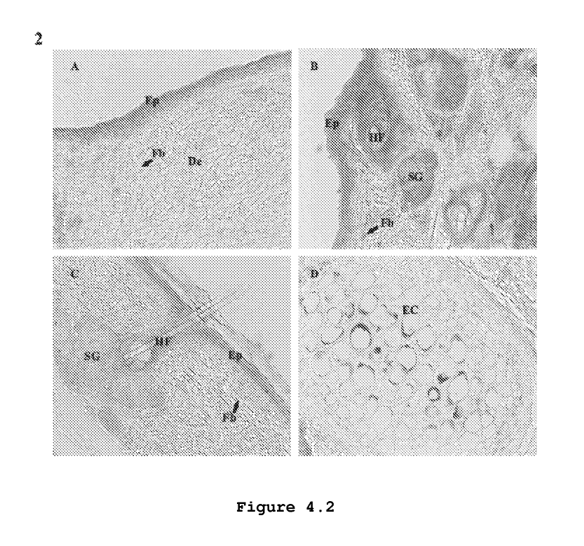

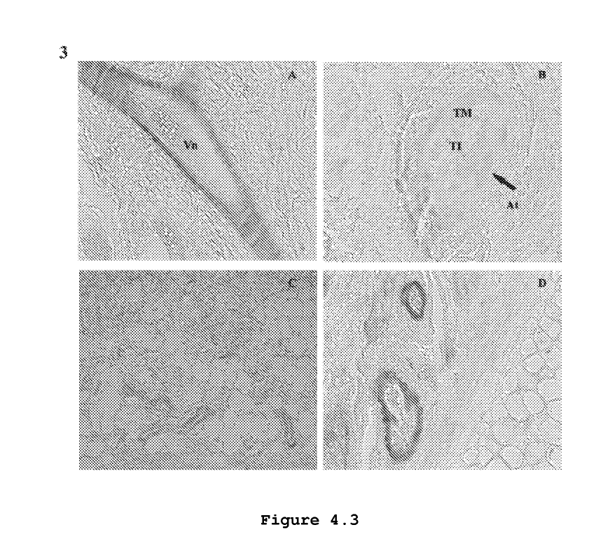

### DETAILED DESCRIPTION OF THE INVENTION

The present invention refers to a new vitamin K-dependent protein with a high content of Gla residues (GRP).

This protein appears to play a key role in modulation of calcification due to its outstanding capability of binding calcium when conveniently gamma carboxylayed, and is expressed and accumulated in almost all tissues tested including soft tissues that do not normally calcify (all types of cartilage, skin and its appendages, vascular and nervous tissues, cardiac muscle, muscle fibers, brain, hematopoietic tissue, blood).

GRP accumulation pattern is wide since it can be found either in the organic extracellular matrix, mainly consisting of glycosaminoglycans or associated to the mineral phase in calcified tissues, namely bone and calcified cartilage, as well as in pathological situations, at sites were ectopic calcification is found, mostly in soft tissues.

It was also verified that the protein is synthesized in different types of cells within these tissues, namely cartilaginous cells in all maturation states, vascular smooth muscle cells, endothelial cells, osteoblasts and osteocytes, fibroblasts, dermatocytes, keratinocytes, neurons, glial cells, leucocytes, cardiac and Purkinje fibbers. Altogether, our in vivo results show that the protein is also associated with soft tissue calcification pathologies at sites of ectopic calcifications, where it is detected co-localized with the mineral, in both vascular system and skin.

For the purpose of using the potential value of GRP, we disclose in detail several methodologies for obtaining GRP from several sources and in different processed forms or functional fragments derived from the intact form, for example, by recovering it from a natural source (also referred as “sample from an organism”) or by chemical synthesis or even from the use of recombinant DNA techniques and suitable cell systems that are well described in the art.

Consequently it is shown that under physiological conditions GRP is one of the most insoluble proteins known and that it easily aggregates.

For the purpose of simplifying the process of isolation and purification of GRP it was also included, as one of the aspects of the present invention, the purification methodologies that make use of valuable biochemical tools as specific antibodies or specific matrices or chemicals, that are also part of this invention, as for example affinity purification techniques based methods, well known to those of skill in the art, or others using specific matrixes and chemicals well described in the examples that are part of this invention.

### 1. Extraction, Isolation and Purification of GRP

For the extraction of GRP from calcified tissues (for example, branchial arches, costal cartilage or bone from mammalian or non mammalian sample organisms), a first extract containing mineral bound proteins can be obtained from calcified ground material using a demineralization procedure (for example a solution containing 10% formic acid or a calcium chelating agent as for example 0.5 M EDTA, pH 8), a dialysis step to remove the dissolved mineral and the dialyzed extract is finally dissolved in denaturant solution for further purification procedures as previously described (Simes, D. C., et al., 2003, 2004 and references therein). For someone well skilled in the art there are well known protein purification techniques that can be use for further purification purposes, as for example, reverse phase high performance liquid chromatography, molecular exclusion chromatography, affinity chromatography or hydroxyapatite mineral matrix in either batch or column formulation.

For the extraction and purification of GRP from non-calcified tissues, several tissues can be used, as for example, skin, blood vessels, and cartilage, from sample organisms (either from mammalian or non mammalian origin, but preferably porcine or bovine). The extraction solution should contain either a strong denaturant, as for example, 4-6 M guanidine, 4-8 M urea, or an extraction buffer (as for example 500 mM NaCl, 50 mM HEPES, pH 7.2, 0.1 mM PMSF or other well described in the literature that concerns protein extraction from cartilage or skin tissues, as for example, described in Belluoccio, D. et al., 2006, Vincourt J. B., et al., 2006, Hannigan A. et al., 2007 and references therein). It is also possible to use additional methods useful for separation of proteoglycans abundant in this type of matrices as for example procedures described by Carrino, D. A., et al. 2003, 1997; Hermansson M., et al., 2004 and references therein. The total extract containing GRP can be further purified using, appropriate and well known in the art, chromatography separation techniques as, for example, described in Examples section.

It is also included in this invention, a method for GRP extraction and purification from biological fluids, preferably blood serum or plasma. For that purpose, blood plasma and/or serum are prepared according to well-established protocols (as for example described in Price, P. A. et al., 2004).

As a possible alternative, a method can be used for a previous separation of high abundant serum or plasma proteins, using for example the ProteoMiner™ Kit (Bio-Rad), according to the manufacture's instructions. The crude serum/plasma sample or the resulting sample after use of the pre-purifying kit is further purified using appropriate, well known in the art, chromatographic separation techniques, as for example the methodologies described in the Examples section.

Cell cultures expressing GRP from several source organisms (preferably, but not limited to, ATDC5, MC3T3-E1, MG63, etc) can also be used for extraction and purification of GRP. For that purpose, the cells are grown on tissue culture plates, according to means known in the art, using appropriate and established mediums, supplemented with 5%-10% of serum, according to the cell culture in use. At 80% confluence, serum free condition media (respective culture medium without serum) is added and after 48 h the resulting conditioned media is collected for further isolation of the secreted protein according to means known in the art, such as by affinity chromatography, gel filtration or reverse-phase chromatography (using, for example, a Vydac C18 reverse-phase HPLC column (4.6 mm id×25-cm length).

GRP can also be extracted from the cells and the secreted organic matrix using a denaturant solution with or without detergent as for example 6M guanidine HCl. Further GRP purification from this extraction solution can be achieved using well known methods in the art such as affinity chromatography, gel filtration or reverse-phase chromatography (on, for example, a Vydac C18 reverse-phase HPLC column (4.6 mm id×25-cm length).

As part of this invention osteoblast-like cell cultures, or others with the ability of producing a mineralized matrix in vitro (preferably but not limited to ATDC5, MC3T3-E1, etc) can be used for isolation of GRP. These cells can be induced to mineralize, using several supplements well known in the art, as for example (β-glycerophosphate and L-ascorbic acid (vitamin C)).

After that, it is possible to separate the GRP bound to the organic extracellular matrix from the GRP that binds the mineral phase. For that purpose it is used first a denaturant solution (as for example 6M Guanidine HCl) and after collected the mineral phase and use a demineralization solution as for example described in Simes et al, 2003, 2004. The extracted proteins can be further purified using well known in the art chromatographic techniques preferably reverse-phase chromatography (on, for example, a Vydac C18 reverse-phase HPLC column (4.6 mm id×25-cm length).

### 2. Production of GRP Using Recombinant Techniques

As part of this invention we can also produce GRP using, well known in the art, recombinant DNA techniques. For this purpose, standard procedures can be performed that include (1) constructing a cDNA encoding the GRP coding sequence or a chimeric fusion protein product, (2) cloning the corresponding cDNA into expression vectors, (3) transforming host cells such as bacteria, yeast, insect, mammalian cells, fish derived cells or plant cells (host systems which are capable of proper post-translational processing are preferred), (4) expressing the cDNA. mRNA coding for GRP can be obtained from a variety of sources as for example, tissues from different organs belonging to the organism of interest, or from cell cultures/lines derived from mammalian, fish or other species of interest once they have been shown to express the GRP gene.

For example, first a DNA encoding the mature protein (used here to include any maturation form) is obtained. For this, the mRNA coding for GRP can be isolated from a variety of sources e.g. tissues, mammalian or fish derived cell cultures, and converted into the corresponding cDNA using well known in the art techniques. As an alternative the complete genomic GRP sequence, e.g. with introns, can be used to transfect mammalian cells. The coding sequence (either cDNA or genomic) is than placed in operable linkage with suitable control sequences in a replicable expression vector. Two, well known in the art, types of systems can be used to express proteins in mammalian cells: those that involve transient or stable expression of transfected DNA and those that involve the use of viral expression vectors derived from simian virus (SV40), vaccinia virus, adenovirus, retrovirus, and baculoviruses (Summers and Smith, 1987).

The vector is used to transform a suitable host, e.g. bacteria, yeast, insect, mammalian cells, fish derived cells or plant cells, and the transformed host is cultured under suitable conditions to induce the production of the recombinant protein.

Many methods, well known in the art, have been developed for introducing eukaryotic DNAs into cultured mammalian cells, as for example, calcium phosphate- or DEAE-dextran-mediated transfection, protoplast fusion, electroporation, liposomes, direct microinjection in the nuclei, among others (fundaments of these techniques are described in detail in Sambrook, J., et al., 1989). Companies have now-a-days developed many new ways of introducing DNA into cells based on that previous knowledge, such as the nucleofection (AMAXA).

To improve the efficacy, solubility and stability of GRP expression, the cDNA may be linked to an extension, encoding a second protein in such a way that expression will give rise to a chimeric fusion protein that can be used at the same time as selective agent.

Several expression vectors are commercially available and can be used as backbone to construct GRP fused or not with another protein. The constructs for expression vectors operable in a variety of hosts should have appropriate replication and control sequences. The control sequences, expression vectors, and transformation methods are dependent on the type of host cell used to express the gene, e.g. bacteria, yeast, insect, mammalian cells, fish derived cells or plant cells. Host systems, well known in the art, which are capable of proper post-translational processing, are preferred. Examples of GRP production are given in the examples section A.

### 3. Production of Peptides Comprising Specific Amino Acid Sequences

The invention also includes several peptides comprising anyone of the contiguous sequences of amino acids set forth in Table 1. These peptides can generally be prepared using methods well known in the art, as for example chemical synthesis or recombinant nucleic acid methods.

### 4. Antibody Production

Antibodies specific for GRP (or a GRP fragment) included in this invention can be produced using conventional methods, including those methods available for producing polyclonal and monoclonal antibodies on a commercial scale, genetically engineered monoclonal antibody or antibodies fragments or antibodies produced by in vitro immunization by certain cells, and by phase display techniques. These methods are well known in the art and described in various well-known laboratory manuals (e.g., Harlow et al., Antibodies: A Laboratory Manual, First Edition (1988) Cold spring Harbor, N.Y.; Harlow and Lane, Using Antibodies: A Laboratory Manual, CSHL Press (1999) and Ausubel, et al., Short Protocols in Molecular Biology, 3rd ed., Wiley & Sons, (1995)).

Preferably in this invention we will use polyclonal antibodies generated in rabbit or goat. Examples of peptides suitable and preferred for immunizations could be any peptide sequence having any contiguous amino acids set forth in any one of the sequences presented in Table 1 namely in regions A, B or even regions A and B. It is also important to refer that for this purpose, in Table 1, any glutamic acid residue (E) could be also used as the modified gamma carboxyglutamic acid residue (Gla).

Certain hybridomas that produce the monoclonal antibodies described in this invention are also in the scope of this invention. Such antibodies may be employed in any of the methods described herein.

As an important part of this invention we include several preferred antibodies (either mono- or polyclonal) that may specifically bind to one of two sequence regions found in GRP protein. These regions are boxed in Table 1, and the region A corresponds to regions of sequence in GRP proteins from different organism species that include all GRP Gla residues and region B correspond to a sequence that do not include any Gla residue. In general, therefore, a subject antibody that could be used in the methods described in this invention can specifically recognize an epitope contained or part of a sequence described in Table 1. This sequence could be part of only one of the regions defined as boxes A or B, or could be part of both of the two regions (A and or B).

Accordingly, since antibodies generally recognize motifs smaller than those contained in region A or B in Table 1, a subject antibody may recognize peptides that are smaller than and contained within the boxed regions (A, B) described in Table 1.

In a preferred embodiment the antibodies may specifically recognize the following antigenes:

(1) peptides homologous to the sequence of a GRP fragment contained in box A (Table 1), as for example, GRP 20-40 (herein named GlaGRP antibody), (2) peptides homologous to the sequence of a GRP fragment contained in shadow box B in Table 1, as for example, and depending on the organism, GRP 54-74 or GRP 58-74 (herein named, CTermGRP antibody) (3) the functional mature GRP purified from a natural source (i.e healthy sample organism) and therefore a gammacarboxylated GRP form (herein named, FunctGRP antibody) (4) a unprocessed non-gammacarboxylated form obtained using well known in the art techniques for preparing recombinant DNA-prepared protein or chemical syntheses-prepared protein (herein named, ucGlaGRP antibody).

The specific antibodies obtained, either mono- or polyclonal, can be affinity purified, using well known in the art methods and validated against the GRP antigen using the corresponding specific antigen forms they were raised against, as for example the mature GRP protein purified from different fish and or mammalian species (for example sturgeon, seabream, bovine, rat, human) as well as against the purified recombinant protein or other GRP processed form or synthetic peptide or protein, using techniques well known in the art as western blot and dot blot analysis.

5. Methods for Detection and/or Quantification of GRP in Samples

It is also described in this invention, assays and methods that can be used for the detection or quantification of either intact or processed forms of GRP in samples such as biological, tissue samples, freshly harvested cells, cell cultures, biopsies, organisms or any of these samples included in paraffin, metacrylate based resins (for example Historesin Plus) or other adequate form of sample preservation for immunohistochemistry analysis.

Accordingly, testing for the presence of GRP may be used in (i) the prognostic and diagnostic evaluation of diseases related to ectopic calcification, (ii) the identification of organisms with a predisposition to these pathologies, (iii) monitoring the progress of ectopic calcification related diseases, and (iv) identifying organisms with calcification pathologies that show resistance to treatments.

Detection and quantification of GRP protein in different processed forms in tissues (for example from biopsies, or other types of tissue samples including histological whole mount preparations/slides, obtained from multiple sources) can be performed using the several procedures described in this invention. As part of this invention it is also described an immuno-histochemical detection method that can be used employing the described preferred specific GRP antibodies (either mono- or polyclonal GRP antibodies) that are also part of this invention.

Methods describing procedures for the detection and/or quantification of circulating GRP in serum or plasma, which includes GRP-related antigen and GRP in different processed forms (as for example different gammacarboxylation status), can be performed using several immunoassay procedures that include the use of the described preferred antibodies, preferably affinity purified antibodies, depending on the species from which the assayed antigen is from, that are also described in this invention.

The antibodies of the invention may be used for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, and cellular immunostaining (fixed or native) assays to name but a few. Such assays are routine and well known in the art (see, e.g., Ausubel et al., eds, 1994, Current Protocols in Molecular Biology, Vol. 1, John Wiley & Sons, Inc., New York, which is incorporated by reference herein in its entirety). Possible immunoassays to be used are described briefly below (but are not intended by way of limitation).

In fact and as part of this invention it is described several types of preferred ELISA-based assays that can be used depending on the availability of a second antibody.

As an example, it is disclosed two single-antibody ELISA assays (competitive ELISA and antibody capture assays) and a sandwich ELISA assay that uses a second antibody. Such immunoassay procedure can result in a qualitative and/or a quantitative analysis, depending on whether or not a quantification method is used. For quantification purposes, dose-response curves should always be prepared with various dilutions of normal pooled serum or plasma. From the data obtained so far we conclude that our assay for serum human GRP is highly reproducible.

The proposed GRP assay is useful as diagnostic tool for both diagnosis and patient follow-up during treatment of several calcification related pathologies. For that, in an embodiment, the invention contemplates a method for monitoring the progression of ectopic calcification in an organism, comprising: (a) testing a sample from the organism to determine the level of GRP in that sample; and (b) repeating step (a) at a later point in time to compare results with those obtained in step (a).

As part of this invention, several different species-specific mRNA probes can be prepared and used for mRNA detection in histological preparations, by in situ hybridization in tissue samples obtained from biopsies, whole organism samples and tissue samples from other origins, in order to evaluate the sites of GRP expression at cell and tissue levels (using well established techniques of in situ hybridization). Quantification and comparative analysis of in situ hybridization results can be achieved, for example, using morphometric analysis. Evaluation and quantification of GRP mRNA levels in either the mentioned tissue samples and/or in blood can also be achieved using the well known technique of real-time Time PC.

Detection of GRP mRNA in histological preparations can be performed using well known in the art techniques of in situ hybridization. Several protocols, commercial available kits for probe preparation, and detection methods can be used. Probes to be used should be species-specific, comprising a region of the mRNA, preferably not less than 200 bp, with or without the 3′, or 5′ UTRs, and should be previously cloned into a suitable plasmid containing T7 and SP6 binding sites, for transcription. Independently of the methodology used, (i) GRP sense and anti-sense probes should be prepared; (ii) samples should be prepared accordingly, (iii) hybridization of samples with GRP probes need to be performed.

Real-time Time PC is one of the most sensitive and easy methods to quantify mRNA levels and can be used for GRP mRNA quantifications from several sources, like for example, from blood, tissues, biopsies, organs, cell cultures, or others. Several real-time Time PC chemistries well known in the art are available and can be used, as for example, taqman probes or similar, fluorescent dyes, like sybergreen, among others. Many real-time Time PC machines, analysis software, and prepared mixes, are in our days commercially available and can be used. For that, and as an example of a conventional procedure: (i) RNA is extracted from the target sample, (ii) cDNA is synthesized by reverse transcription (RT) and either specific GRP and normalizing gene primers, as universal dT adapter, (iii) Time PC is performed according to the chemistry in use, (iv) quantification is achieved after analysis and interpretation of the results using a real-time specific software.

Accordingly, the detection and quantification of GRP mRNA levels may be used in the prognostic and diagnostic evaluation of diseases related to ectopic calcification.

### Examples

A. Isolation of GRP from Several Sources

The following are typical examples illustrating the preparation of GRP extract, subsequent purification from several sources (for example, tissues, organs, organisms, biological fluids, tissue culture, cell culture, recombinant sources), the preparation of monoclonal and polyclonal antibodies specifically recognising epitopes and/or conformations on GRP protein, methods for the detection and or quantification of GRP protein processed forms in several samples, and several other aspects.

A1. GRP Preparation from Calcified Tissues

Material preparation: Fresh calcified materials (for example, branchial arches, costal cartilage or bone) are clean from adhering soft tissues, lyophilized and reduce to a fine powder. The material is extensively washed at 4° C. with vigorous stirring, 3 times with 10-fold excess of 6M Guanidine followed by water and acetone. After, the material is allowed to dry overnight.

Protein extraction: mineral bound proteins are extracted using a 10-fold excess of 10% formic acid for 4 hours at 4° C. as described (Simes et al., 2003). The extracted proteins are separated from the insoluble collagenous matrix by filtration through filter paper and next dialyzed at 4° C. against 50 mM HCl using 3500 molecular weight tubing (Spectra-Por 3, Spectrum, Gardena, Calif.) with 4 changes of medium over 2 days to remove all dissolved mineral. The dialyzed extract is freeze-dried, dissolved in 6 M guanidine HCl, 0.1 M Tris, pH 9, and dialyzed against 5 mM ammonium bicarbonate to precipitate GRP. A portion of this solution was dialyzed against 50 mM HCl, dried, and aliquots of the resulting protein precipitate were analyzed by SDS-PAGE. Protein profile was revealed by staining, either with CBB (Bio-Rad, Richmond, Calif.) or DBS (Gla-specific stain; 8.5 mM 4-diazobenzene sulfonic acid (Sigma, Spain), as described (Jie, K. S., et al., 1995). The crude precipitate is further purified by reverse phase high performance liquid chromatography (RPLC) in order to obtain pure GRP as described further in this section.

Protein purification from crude extract: For further purification several well-known purification methodologies can be used and here we described as an example several preferred chromatographic methods:

a) Reverse-phase FPLC technique: the crude protein precipitate obtained after direct extraction with 6 M guanidine HCl, 0.1 M Tris pH 9 or using other protein extraction procedures are divided in 250 microliters to 1000 microliters aliquots and injected, for example, onto a Vydac C18 reverse-phase HPLC column (4.6 mm id×25-cm length) equilibrated in 0.1% trifluoroacetic acid in water and at a flow rate of 1 ml/min (initial conditions). The HPLC are run for 7 min at initial conditions, and then proteins are eluted from the column with a 1.5 h linear gradient to 0.1% trifluoroacetic acid in 60% acetonitrile. Fractions of 1 ml are collected and their absorbance determined at 220 nm. Further purification can be achieved using a molecular exclusion chromatography as follows in b).
b) Molecular exclusion chromatography: a column is packed with an adequate resin as for example Sephadex G-75 and fractions from reverse-phase FPLC purification containing GRP are freeze-dried, re-dissolved in a small volume of for example an acid solution (i.e. 0.5 ml of 50 mM HCl) and purified over a 25-ml Sephadex G-75 column equilibrated in the same buffer (i.e 50 mM HCl). Fractions of 0.5 ml are collected and absorbance determined at 220 nm.
c) Affinity purification of GRP can be performed using one of the well described methods available (for example see: Antibodies, a laboratory manual by E. Harlow and D. Lane, 1986) for (1) the preparation of the antibody affinity column that means selecting an appropriate matrix, from the several commercially available, and coupling the selected antibody to the bead matrix (using either monoclonal or polyclonal purified selected antibodies that are also described in this invention) (2) Eluting antigen from immunoaffinity column that can preferably be done using an acid solution, pH 3-1.5 or Guanidine solution 2-5 M, urea 2-8 M, or other suitable solution as described in: Antibodies, a laboratory manual by E. Harlow and D. Lane, 1986.
d) Hydroxyapatite matrix material (commercially available) in either column or batch can also be used for the separation of this Gla rich protein using the high affinity of the protein for calcium mineral surfaces.

Batch methodology includes incubation of the sample solution or extract containing GRP directly (as for example plasma or serum or cell culture medium) with the hydroxyapatite matrix material using an aqueous solution buffer pH 5-12 (that might also contain other components as for example a detergent, a denaturant, an inorganic salt) followed by cleavage of the protein-mineral bond using a demineralization solutions containing an acid or a calcium chelating agent (for example 5-10% formic acid or 0.1-0.5-Methylenediaminetetraacetic acid, EDTA, 10 times de volume). After dialysis in acidic conditions to remove the dissolved mineral, further purification of the solution containing Gla-proteins (including GRP) that bind hydroxyapatite can be performed as described in (Simes et al, 2003) and final purification can be performed using one of the chromatographic methods described before.

A2. GRP Preparation from Non-Calcified Tissues

Material preparation: The materials (skin and cartilage from sample organisms, preferably porcine or bovine) are first lyophilized and reduce to a fine powder. From calculated weight a 10-fold excess of extraction solution consisting of 6M Guanidine HCl is added with vigorous stirring at 4° C. for 24 hours. After, the solution cleared by filtration through filter paper is directly applied in a FPLC column for chromatographic separation as described before in examples A1 a) and b).

A3. GRP Preparation from Biological Fluids (for Example Blood)

For serum preparation fresh blood is collected and allowed to clot for 30 min at room temperature, centrifuged at 5000 rpm and the serum collected; for plasma preparation, fresh blood is collected and anticoagulants, like for example heparin, can be added to avoid clotting, centrifuged at 5000 rpm and the plasma collected. High abundant serum or plasma proteins can be first separated from low abundance serum proteins using for example ProteoMiner™ Kit (Bio-Rad), according to the manufacture's instructions. The resulting solution is directly applied (250 μl-1000 μl) to a reverse phase FPLC column as described in A1 a) and b).

A4. GRP Isolation from Cell Culture

### A4.1 Cell Cultures Preparation

ATDC5 cells: 5 plates were cultured in differentiation medium [(DME/Ham's 12 (1:1), Invitrogen, with 5% FBS (Sigma), 1% penicillin/streptomycin (Invitrogen), and 1× insulin, transferine and sodium selenite (ITS) (Invitrogen)], at 37° C. in 5% CO₂, for 21 days. To induce mineralization ATDC5 cells were transferred to a calcification medium [α-MEM (Invirogen) with 5% FBS (Sigma), 1% penicillin/streptomycin (Invitrogen), and 1×ITS (Invitrogen)], at 37° C. in 5% CO₂ for more 15 days. MC3T3 cells: 10 plates were cultured in calcification medium [α-MEM (Invirogen) with 10% FBS (Sigma), 1% penicillin/streptomycin (Invitrogen), 10 mM β-glycerophosphate, 50 μg/ml L-ascorbic acid and 7 μg/ml of vitamin K1 (Sigma)], at 37° C. in 5% CO₂, for 5 weeks.

In all cell cultures mediums were changed every 2 days. Mineral deposition was always revealed using von Kossa staining as described (Pombinho et al, 2004).

A4.2 GRP Extraction from Cell Cultures Medium

Medium was collected from ATDC5 cultures at the end of days 21 and 36. From MC3T3 cultures, the medium was collected at the end of the 5 weeks of mineralization experiment. The collected medium was centrifuged for 10 min at 13000 rpm, and filtered through a 0.2 μm filter. The resulting cell debris free medium was dialyzed (SpectraPor 3) against 50 mM HCl, over 48 h with 4 changes of serum-free medium, freeze-dried, re-suspended in 50 mM HCl. The 50 mM HCl solution containing GRP is directly applied (250 μl-1000 μl) to a reverse phase FPLC column as described in A1 a) and b).

A4.3 GRP Extraction from the Cells and Secreted Organic Matrix

ATDC5 cells at the end of day 21 (or 36) and MC3T3 cells at the end of the 5 weeks of the mineralization experiment were scraped with 6 M guanidine HCl, 0.1 M Tris pH 9. The extract was washed twice with 6 M guanidine HCl, 0.1 M Tris pH 9 for 8 hours at 4° C. and centrifuged at 10,000 rpm for 15 min:
a. Proteins extracted with 6 M guanidine HCl, 0.1 M Tris pH 9 (includes the intracellular proteins and also the proteins bound to the extracellular organic matrix) were dialyzed (SpectraPor 3) against 50 mM HCl, over 48 h with 4 changes of medium, freeze-dried and re-suspended in 50 mM HCl. The presence of GRP was confirmed trough western blot using the CTermGRP antibody. The 50 mM HCl solution containing GRP is directly applied (250 μl-1000 μl) to a reverse phase FPLC column as described in A1 a) and b).
b. The mineral nodules from the MC3T3 cells were pellet in the above described centrifugation and GRP was extracted as follows:
   A4.4 GRP Preparation from In Vitro Calcified Nodules

The pellet containing the mineralized nodules was demineralized with 10% formic acid (v/w) at 4° C. for 4 hours. The resulting acid extract was dialyzed at 4° C. against 50 mM HCl using a 3500 molecular weight cut-off tubing (SpectraPor 3) with four changes of the medium over two days in order to remove all dissolved mineral. The entire dialyzed extract was freeze-dried and re-suspended in 50 mM HCl. The presence of GRP was confirmed trough western blot using the CTermGRP antibody. The 50 mM HCl solution containing GRP is directly applied (250 μl-1000 μl) to a reverse phase FPLC column as described in A1 a) and b).

The following are typical examples illustrating the production and purification of recombinant GRP

### A5. Preparation of Recombinant GRP

1 μg of total RNA extracted from human skin was reverse transcribed with MMLV-RT (Invitrogen), and human GRP cDNA was Time PC amplified using human GRP specific primers (HGRPF1 and HGRPR1 to obtain HuGRP-pcDNA3.1, and HGRPF2 and HGRPR2 to obtain Ile-Glu-Gly-Arg-GRP-DHFR (for details see below)), and Advantage Taq DNA polymerase (BD Biosciences). The amplified cDNA was first cloned into pCR^{II}TOPO (Invitrogen), sequenced to obtain one positive and non-mutated clone, and then sub-cloned into pcDNA3.1 (Invitrogen), and pQE40 (Qiagen) vectors through digestion of the HuGRP-pCR^{II}TOPO and vectors with appropriate restriction enzymes, and ligated using T4 DNA ligase (Invitrogene).

HGRPF1 (5′-3′):
ACCTCTGCAAAGATGACTTGGAGAC,
and
HGRPR1 (5′-3′):
GATCACGTGTGGTGGCGGTTGTAGA.
HGRPF2 (5′-3′), for SphI restriction site at
5' end of human GRP:
TATGCATGCATTGAAGGTCTCCCCCAAGTCCCGAGATGA;
HGRPR2 (5′-3′),
for PstI restriction site at the 3' end of human
GRP:
TATCTGCAGCGTGTGGTGGCGGTTGTAGA

### A5.1. Preparation of Recombinant GRP in Mammalian Cells

HuGRP-pCR^{II}TOPO and pcDNA3.1 (Invitrogen) were digested with EcoRI restriction enzyme (Takara), for 2 h at 37° C., loaded into a 2% agarose gel, extracted and purified from the gel with GFX Time PC DNA and gel band purification kit (Amersham Biosciences). Ligation of the GRP coding sequence to the pcDNA3.1 expression vector was achieved using T4 DNA ligase (Invitrogene) for 2 h at room temperature. DHL-5α (Invitrogen) was transformed with the ligation product and positive clones were sequenced for sequence and orientation confirmation. Only clones with HuGRP coding sequence, which includes GRP ATG start codon, cloned in frame with the origin of the plasmid were selected. HuGRP-pcDNA3.1 clone was transfected into ATDC5 cells (RIKEN cell bank (Tsukuba, Japan) number: RCB0565) using the Fugene reagent (Roche), as transfection reagent, according to manufacture's instructions. Stable transfectants were selected using G418 sulfate, and used to overexpress human recombinant GRP. γ-carboxylation can be stimulated or blocked using vitamin K or sodium warfarin respectively. To stimulate carboxylation cells are grown in their respective medium supplemented with 10% charcoal-stripped serum, antibiotics and 7 μg/ml of vitamin K1. To block carboxylation, the cells are grown in their respective medium supplemented with 10% charcoal-stripped serum, antibiotics and 50 μM sodium warfarin, and allowed to expand. In both cases either the medium as the matrix produced by the cells can be collected after confluence, and the secreted γ-carboxylated or non-γ-carboxylated GRP can be further extracted and purified as described in the A4 section. Expression experiments were followed by analysis of the product by SDS-PAGE gel electrophoresis and Western-blotting, with cross-reaction with CTermGRP antibody.

### A5.2. Preparation of Non-γ Carboxylated GRP in Bacteria

For cloning and expression of the chimeric construct of murine dihydrofolate reductase and GRP (DHFR-GRP) the commercially available QIAexpress system was used (Qiagen Inc.). Fusion proteins were constructed using the pQE40 vector, which contains an expression cassette consisting of the phage T5 promotor fused to the mouse DHFR protein. To facilitate protein purification by metal chelate affinity chromatography the recombinant protein was engineered to contain an in frame six residues long histidine (6-His) tail preceding the DHFR.

The HuGRP-pCR^{II}TOPO was used as template in a Time PC reaction with HuGRPF2 and HuGRPR2 primers, containing 5′-SphI and 3′-PstI digestion sites, respectively. The amplified cDNA was digested with SphI and PstI (Takara) and the resulting fragments were then separated by agarose gel electrophoresis and isolated thereof using GFX Time PC DNA and gel band purification kit (Amersham Biosciences).

The isolated fragments and linearized pQE40 vector were then ligated with T4 DNA ligase (Invitrogen), for 2 h at room temperature, and subsequently transformed into E. coli strain M15[pREP4]. For the identification of clones expressing a 6H is tagged DHFR protein fused to GRP the colony blot procedure was used according to the QIA manual instructions. Positive clones were searched through sequencing and selected if they were in the correct reading frame. The construct was transformed into E. coli strain BL21 [DE3, pREP4], which is deficient in the Ion and ompT proteases. Selection of recombinant protein producing clones through the colony blot procedure and using the 6His monoclonal antibodies demonstrated several GRP producing clones. Expression experiments were followed by analysis of the product by SDS-PAGE gel electrophoresis and Western-blotting, with cross-reaction with 6-His monoclonal antibodies (Sigma) and CTermGRP antibody.

### B. Immuno-Based Assays

### B1. Preparation of Monoclonal or Polyclonal Antibodies

### Monoclonal Antibodies

The techniques here described to develop monoclonal antibodies are known in the art see, e.g., Antibodies, a laboratory manual by E. Harlow and D. Lane, 1986).

Balb/C mice are immunised intraperitoneally with one of the designed synthetic peptides homologous to (i) the N-terminus of GRP (residues 20-40 in Table 1), (ii) the C-terminus of GRP (residues 54 or 58-74 depending on the species and represented shadowed in gray in Table 1), or (iii) a synthetic peptide homologous to an internal GRP fragment (amino acid sequences used for raising antibodies are described in table 1), which are coupled to keyhole limpet hemacin (KLH from Pierce, product nr. 77107). The antigen is mixed with Freund's complete adjuvant and used for the first immunisation. The animals are boosted every second week with antigen in Freund's incomplete adjuvant according to well-known immunization protocols. Post-immune sera is screened with an indirect ELISA using purified antigens according with protocols described in well known laboratory manuals (e.g., Antibodies, a laboratory manual by E. Harlow and D. Lane, 1986).

### Polyclonal Antibodies

The techniques used to develop polyclonal antibodies are known in the art (see, e.g., Methods of Enzymology, “Production of Antisera With Small Doses of Immunogen: Multiple Intradermal Injections”, Langone, et al, eds. (Acad. Press, 1981)).

The same antigens described for the production of monoclonal antibodies (any sequence contained and described in Table 1 or a corresponding homologous synthetic peptides or corresponding complete protein in different processed forms obtained either from natural source or using adequate DNA recombinant techniques) can be used for the production of polyclonal antibodies that are also part of this invention.

Accordingly, the described preferred antigens that recognise the sequences described in Table 1 are mixed with an adjuvant, and a suitable non-human animal (e.g., a mouse, chicken, goat, rabbit, hamster, horse, rat or guinea pig, etc.) is immunized using standard immunization techniques (e.g., intramuscular injection) and once a specific immune response has been established (as determined by dot blot or other suitable analytical procedure), blood is drawn from the animal and polyclonal antisera that specifically binds to described peptides isolated and screened with an indirect ELISA as described. Purification of said antibodies is done using affinity purification techniques, in most cases we used the CNBr activated Sepharose resin (Amersham), as described in: Antibodies, a laboratory manual by E. Harlow and D. Lane, 1986

### B2. Methods and Assays for Detection of GRP

### B2.1 Western and Dot Blot

Western blot analysis generally involves preparation of protein samples followed by electrophoresis of the protein samples in a polyacrylamide gel (e.g., 4%-12% SDS-PAGE depending on the molecular weight of the antigen), and transfer of the separated protein samples from the polyacrylamide gel to a membrane such as nitrocellulose, PVDF or nylon. Following transfer, the membrane is blocked in blocking solution (e.g., PBS with 3% BSA or non-fat milk), washed in washing buffer (e.g., PBS-0.05% (v/v) Tween 20), and incubated with primary antibody (the antibody of interest raised against the specific antigens described in Table 1) diluted in blocking buffer. After this incubation, the membrane is washed in washing buffer, incubated with a secondary antibody (which recognizes the primary antibody, e.g., an anti-rabbit antibody) conjugated to an enzymatic substrate (e.g., horseradish peroxidase or alkaline phosphatase) or radioactive molecule (e.g., 32P or 125I), and after a further wash, the presence of the antigen may be detected.

A person skilled in the art would be knowledgeable as to the parameters that can be modified to increase the signal detected and to reduce the background noise. For dot blot assays we deposit the sample directly in the membrane such as nitrocellulose, PVDF or nylon and after we follow the described procedure for specific binding to antibody and detection. The procedure that was more often used is described in detail in (Simes D. C. et al, 2003, 2004).

### B 2.3 ELISA-Based Immunoassays

Two procedures for the GRP assay were worked out:

Antibody-capture ELISA: Allows the detection, in each assay, of either (1) total GRP in the sample analyzed using CTermGRP antibody (2) non-gammacarboxylated GRP using ucGlaGRP antibody.
Sandwich ELISA: Allows the recognition of specific processed molecules only (for example detection and quantification of gammacarboxylated GRP, if we use the specific CTerm GRP as primary antibody and, GlaGRP antibody as a second antibody).

All the methodologies described here were developed based on well-described immunobinding assays of the type generally described in: Antibodies, a laboratory manual by E. Harlow and D. Lane, 1986.

### B 2.3.1 Antibody-Capture ELISA.

This type of assays can be used to detect and quantify antigens and also to compare epitopes recognized by different antibodies. The general protocol is simple: a known excess of antibody is mixed with the test solution containing an unknown amount of antigen and the mixture is added to a microtiter plate coated with antigen. Non-bound antibody from the test sample will be bound to the microtiter well, and will be quantified with a second (labelled) antibody.

The procedure here describe is for the use of either CtermGRP or ucGlaGRP as capture antibodies but other combination are also applicable and is chosen based on the assay purpose.

Coating: Urea-solubilized recombinant GRP is diluted 50 fold with coating buffer (0.1 M sodium carbonate buffer, pH 9.6) and used for coating the PVC microtiter plates with an excess of antigen (50 microliters of purified antigen solution in each well, 10 micrograms/ml), the plates being then allowed to stand for 2 h at room temperature in a humid atmosphere and the wells are wash 2×10 min with PBS/Tween 20 buffer (phosphate-buffered saline containing 0.05%, v/v Tween 20).

Remaining active sites are blocked filling the wells to the top with blocking buffer (3% BSA in PBS/0.05% Tween 20 buffer), for 2 h at room temperature. Wash the plates 2×10 min with PBS/Tween 20 buffer (phosphate-buffered saline containing 0.05%, v/v Tween 20).

Sample preparation: Test samples (preferably serum or plasma samples) are supplemented with a known concentration of antibody (diluted in 3% BSA/PBS) and incubated for 5 min at room temperature.

Assay: The samples (50 microliters) are then transferred to the microtiter plates and incubated for 2 h at room temperature. After washing 3×10 min with PBS/Tween 20 buffer (see above) we add the second antibody: rabbit anti-mouse total IgG labelled with horse radish peroxidase diluted in PBS-Tween buffer) for TMB staining. After washing 3×10 min with PBS/Tween 20 buffer, 0.1 ml TMB substrate is added (TMB substrate kit, Pierce) and incubated at room temperature for 30 min-1 hour. The reaction finishes by the addition of a solution of 1M H₂SO₄. The plate is then read at 450 nm using an ELISA plate reader.

### B 2.3.2 Sandwich ELISA

In this type of assays we use two antibodies, each directed for different epitopes on the antigen. One antibody is bound to the solid plate and the antigen in the sample is allowed to bind. Unbound antigen is washed and a labeled second antibody is added for binding to the antigen. The procedure we described here is for antibodies CTermGRP and GlaGRP but other combination are also applicable and is chosen based on the assay purpose. With this described specific assay we can determine and quantify the amount of gammacarboxilated GRP in the sample. The assay is quantifiable by measuring the amount of bound labeled second antibody.

Coating: Purified CTerm GRP antibody (100 microliters per well (5 micrograms/ml in 0.1 M sodium carbonate buffer, pH 9.6) is immobilized. Incubation is performed for 2 hr at room temperature in a humid atmosphere and the wells are washed 2×10 min with PBS/Tween 20 buffer (phosphate-buffered saline containing 0.05%, v/v Tween 20). After we fill the wells to the top with blocking buffer (3% BSA in PBS/0.05% Tween 20 buffer), for 2 h at room temperature and wash the plates 2×10 min with PBS/Tween 20 buffer (phosphate-buffered saline containing 0.05%, v/v Tween 20).

Sample preparation: 50 microliters of test samples (preferably serum or plasma samples) are added to the wells. For quantification, the antigen solution should be titrated. All dilutions are done with blocking buffer. After, we incubate for at least 2 hours at room temperature in a humid atmosphere. Plates are then washed 4×10 min with PBS/Tween buffer

Assay: The labeled second antibody (in this specific case we used 100 microliters of biotinylated GlaGRP purified antibody (5 micrograms/ml), is added to each well and in general the amount of antibody should be determine in preliminary experiments and all dilutions should be done in blocking buffer. After, we incubate for at least 2 hours at room temperature in a humid atmosphere and wash 3×10 min with PBS/Tween buffer. For detection, we add 0.1 ml of freshly prepared TMB substrate (TMB substrate kit, Pierce) to each well and incubated at room temperature for 30 min-1.hour. The reaction is finished by the addition of a solution of 1M H₂SO₄. The plate is then read at 450 nm using an ELISA plate reader.

### B 2.3.3_Making the Assay Quantitative

To quantify the levels of antigen in different sample we prepare serial dilutions of each antigen test solution in blocking buffer and perform the rest of the assay as described. To determine the absolute amounts of antigen we compare the midpoints of the titration curves. To determine the absolute amount of antigen we compare these values with those obtained using known amounts of the pure antigen for the corresponding specific antibody in use (synthetic peptide, recombinant GRP or purified GRP from natural source).

C. The Following are Typical Examples Illustrating the GRP mRNA Detection and/or Quantification in Histological Preparations, and/or Tissue Samples, Using Species-Specific mRNA Probes and Real-Time Time PC.
C1.1 Species-Specific mRNA Probes can be Used for Detection, by In Situ Hybridization.

### C1.1.1 Probe Preparation

Specific sequences from selected organisms (for example human, rat, mouse, sturgeon) i.e. complete or partial coding sequences, 5′ or 3′ untranslated sequences, can be cloned into appropriate plasmids (as for example pCR^{II}TOPO, Invitrogen), linearized with suitable restriction enzymes, and transcribed with SP6 or T7 RNA polymerases to generate antisense and/or sense riboprobes. Probes are then labeled with for example digoxigenin using preferably but not limited to the RNA labeling kit (Roche, Mannheim, Germany) according to manufacturer's instructions. As an example, a 417-bp fragment of rat GRP cDNA (spanning from nucleotide 417 to the 3′ end) cloned in pCRII-TOPO was either linearized with ApaI and transcribed with SP6 RNA polymerase to generate an antisense riboprobe, or linearized with KpnI and transcribed with T7 RNA polymerase to generate a sense riboprobe. Probes were then labeled with digoxigenin using RNA labeling kit (Roche, Mannheim, Germany) according to manufacturer's instructions.

### C1.1.2. Samples Preparation

Samples from either healthy or target diseased tissues, or tissues from other sources, are collected in freshly prepared sterile 4% paraformaldehyde solution, at 4° C., dehydrated with increasing methanol concentrations and embedded in paraffin or other suitable polymer/resin. If necessary, and prior to embedding, tissue samples can be decalcified for appropriate periods of time in, for example, sterile buffered EDTA (0.3 M EDTA, 0.15 M NaCl, 0.1 M Tris-HCl, pH 7.6). Once embedded, samples are sectioned (6-8 μm thick) and collected in, for example, TESPA (3-aminopropyltriethoxysilane, Sigma) coated slides. In the case of calcified tissues being used for GRP detection by immunohistochemistry, these tissue samples must be processed without previous decalcification, and in this case they are embedded in, for example, HistoResin plus basic resin prior to sectioning with the appropriate microtome.

### C1.1.3. Hybridization

Several hybridization protocols are well established and available in the literature (as described in detail in for example Sambrook, J., et al., 1989). Briefly, and as an example and not a limitation, sections are digested with proteinase K (Sigma) (40 μg/ml) in 1×PBS (phosphate-buffered saline) containing 0.1% of TWEEN 20 (Sigma) (PTW) for 15-45 min then hybridized at a temperature between 60°-68° C. overnight in a humidified chamber. After hybridization, sections are washed and signal revealed with, for example, the alkaline phosphatase-coupled antidigoxigenin-AP antibody (Roche) and NBT/BCIP substrate solution (Sigma).

### C1.1.4. Analysis of the Results

Quantification and comparative analysis can be achieved, for example, using morphometric analysis and evaluation performed by an expert in the field.

C2. Quantification of GRP mRNA Levels by Real-Time Time PC

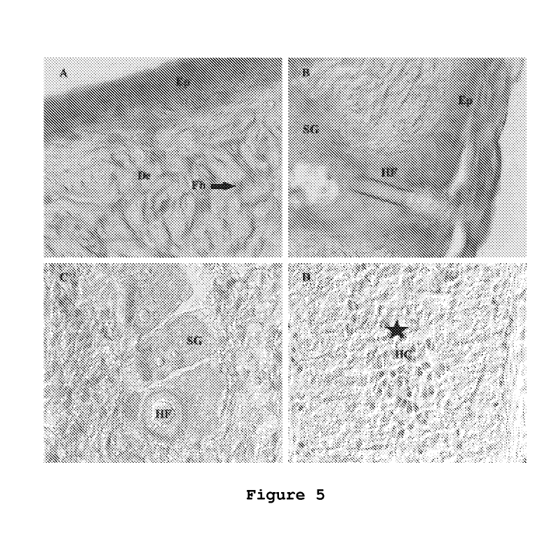

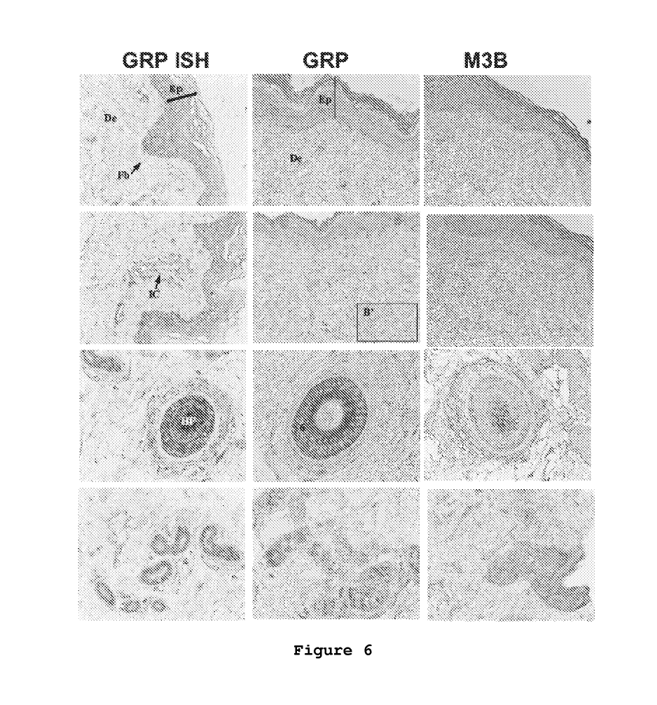

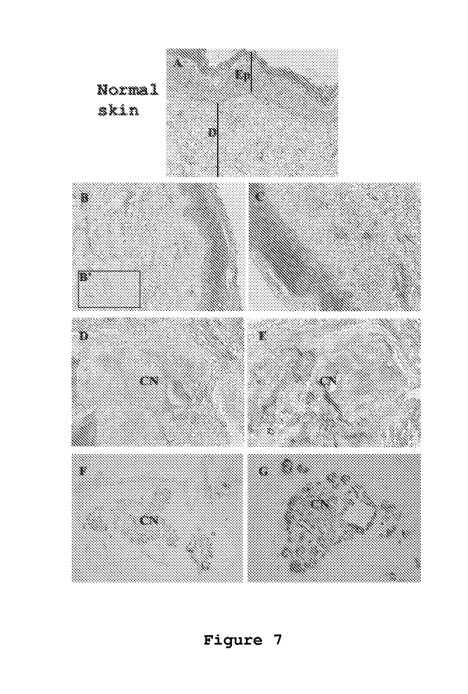

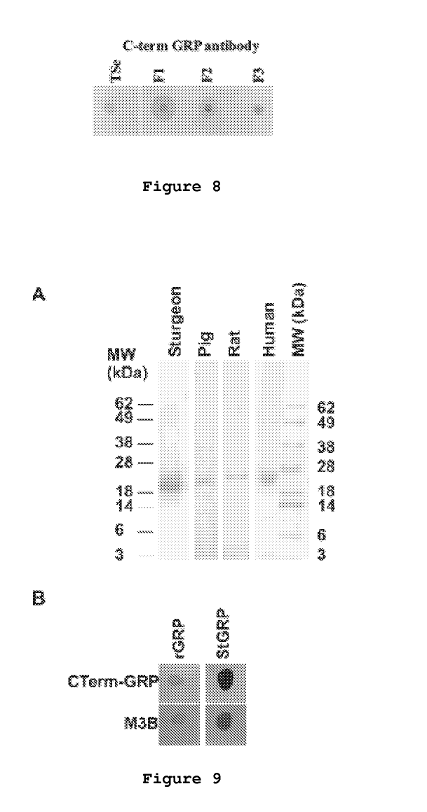

Evaluation and quantification of GRP mRNA levels in tissue samples obtained from biopsies, whole organism samples, blood, cell cultures and tissue samples from other origins can be achieved using the well-known technique of real-time Time PC. As an example, and not a limitation, the relative expression of GRP was determined in several tissues from sturgeon and rat (FIG. 5), using Absolute QTime PC SYBR Green Fluorescein mix (ABgene, Epsom, UK) in an iCycler iQ apparatus (Bio-Rad). One microgram of total RNA of each tissue was treated with RQ1 RNase-free DNase (Promega, Madison, Wis.) and reverse-transcribed at 37° C. with MMLV-RT (Invitrogen) using universal dT-adapter as a reverse primer. Rat and sturgeon GRP were amplified with species-specific primers and glyceraldehyde-3-phosphate dehydrogenase (GAPDH) and hypoxanthine phosphoribosyltransferase 1 (HPRTI) were used as control genes. Fluorescence was measured at the end of each extension cycle in the FAM-490 channel and melting profiles of each reaction were performed to check for unspecific product amplification. Levels of gene expression were calculated using the comparative method (ΔΔCt) and normalized using gene expression levels of GAPDH or HPRTI housekeeping genes.

### D. The Following are Typical Examples Illustrating the Detection of GRP Protein Accumulation in Different Processed Forms Using Species-Specific GRP Antibodies, in Histological Preparations

### D1. GRP Specific Antibodies can be Used to Detect Protein Accumulation in Histological Preparations, by Immunohistochemistry

Samples collected for in situ hybridization can be used for evaluation of GRP accumulation either from healthy or target diseased tissues, or any tissues of interest from other sources.

Several immunohistochemistry protocols are available and can be used (for example as described in detail in Sambrook, J., et al., 1989) to detect GRP accumulation. Briefly and as an example and not a limitation, after deparaffination, the endogenous peroxidase activity is blocked with 3% H2O2 in Coons buffer (CBT: 0.1 M Veronal, 0.15 M NaCl, 0.1% Triton X-100) and, if GRP is to be detected within cartilaginous matrix, paraffin sections are pretreated with, for example, testicular hyaluronidase or chondroitinase for 1 h at 37° C.

Nonspecific binding to sections is blocked with 0.5% (wt/vol) bovine serum albumin (BSA) in CBT for at least minutes. Incubation with specific primary purified polyclonal or monoclonal antibodies (for example, CTermGRP, GlaGRP or ucGlaGRP antibodies) is performed overnight in a humidified chamber at room temperature.

After several washes in CBT, sections are incubated in the same buffer for 1-2 h at room temperature with, for example, peroxidase-labeled goat anti-rabbit IgG (Gibco-BRL) secondary antibody at manufacture's recommended dilutions. Peroxidase activity is revealed using 0.025% 3,3-diaminobenzidine (Sigma). Quantification and comparative analysis can be achieved, for example, using morphometric analysis and evaluation can be performed by any expert in the field.

E. Screening for GRP Mutations and/or Polymorphisms

Screening for GRP mutations and/or polymorphisms in the coding region can be detected using Time PC amplified regions from genomic DNA, or from cDNA by using sequence-specific primers and preferably a high fidelity polymerase (such as Clontech HF-2) to avoid polymerase-introduced mutations. Screening for GRP mutations and/or polymorphisms in regulatory and intronic regions of the GRP gene can be performed by Time PC amplifying regions from genomic DNA using sequence-specific primers and preferably a high fidelity polymerase (with proof-reading capacity). Amplified Time PC fragments can be directly sequenced with the corresponding set of species-specific primers, and analyzed for the presence of mutations and/or polymorphisms by sequence alignment with the established wild type sequence.

This method can be used to detect multiple deletions, insertions, substitutions, and single nucleotide polymorphisms (SNPs) in the GRP gene, either in a given population or in specific individuals. Several well established methods have been developed and published for genomic DNA and RNA extraction as well as for cDNA synthesis from RNA template (as described in Sambrook, J., 1989), and many commercial kits are now a days available to perform these type of procedures (as for example PAXgene blood DNA kit (Qiagen), EZ1 Tissue Mini kit (Qiagen), MMLV-RT (Invitrogene)).

As an example and not a limitation, genomic DNA can be isolated from blood, or any tissue source from a given patient and individual coding exons, individual introns, promoter, or the complete gene can be Time PC amplified using a high fidelity polymerase and sequence specific primers. For example, the following set of primers can be used to amplify individual GRP exons and splice sites: exon 1: HsEx1_{—}1F: 5′-TCTTCCCTCCCCCGCCTTCCTT-3′ and HsEx1_{—}1R: 5′-TTTGTTTCTTTCTTTACTGTTTTTTAT-3′ or HsEx1_{—}2F: 5′-CTCCTCCTCTCCCCCAGTGGTATC-3′ and HsEx1_{—}2R: 5′-TAACAATAAGCATACTCCTTTCTACC-3′; exon 2 and exon 3: HSEx2-Ex3_{—}1F: 5′-GTGGGTTCCTGGGGAGATTGGCT-3′ and HSEx2-Ex3_{—}1R: 5′-GCCTCCTAACCCTGACAAAGTATTC-3′; exon 4: HsEx4_{—}1F: 5′-TGACTTCTCAGATGAACTGTGCTCC-3′ and HsEx4_{—}1R: 5′-AAAGTTGGGTAGAAGAAGAGAAAGC-3′; and exon 5: HsEx5_{—}1F: 5′-CACCAGGGCTCACAAACACTCTC-3′ and HsEx5_{—}1R: 5′-TCACATCATCGCTCAGGGAAGACAG-3′. Time PC products are then directly sequenced using the corresponding set of specific primers, to obtain the sequence of both strands for the 5 exons of the GRP gene, and corresponding splice sites. The promoter region(s) can be amplified using, for example the following set of primers: HsGRP_Pr1F: 5′-TAAATAGACATGGGGGTCTCGCTA-3′ and HsGRP_Pr1R: 5′-ATCTTTGCAGAGGTAGGGGCTCCG-3′, and directly sequenced with the same specific primers. The complete gene can be amplified and sequenced using, for example, the following set of primers: HsGRP_Pr1F: 5′-TAAATAGACATGGGGGTCTCGCTA-3′ and HsEx5_{—}1R: 5′-TCACATCATCGCTCAGGGAAGACAG-3′. If mutations and/or polymorphisms are within the coding region, one can use RNA transcribed to cDNA to Time PC amplify in a single fragment the complete coding sequence, using for example the following set of primers:

| | |
|---|---|
| HsGRPF: | 5′-AGGACGCCTGGTCTGCCTTGTGGGT-3′, |
| and | |
| HsEx5_1R: | 5′-TCACATCATCGCTCAGGGAAGACAG-3′. |

Alignments of the obtained fragment sequences, with the wild type sequence, allows to identify and detect multiple deletions, insertions, substitutions, and SNPs in the GRP gene of the patient in study.

### DEFINITIONS

The word GRP refers to a vitamin K dependent protein containing high density of Gla residues (22%) therefore, stating for a Gla-rich protein.

The term “organism” as used herein means a human, a non-human mammal, or a non-mammalian specimen. In a preferred embodiment, the organism is a mammal.

The term “sample from the organism” as used herein means any sample that might contain mature GRP or that one wishes to analyze for presence of GRP including, but not limited to, biological fluids, tissue extracts, freshly harvested cells, and lysates of cells, which have been incubated using in vitro cultures. In a preferred embodiment, the sample is serum or plasma obtained from an organism.

The term “soft tissue” refers to a tissue that is not calcified in a normal healthy organism.

The terms “calcification” refers to the deposition of calcium in a tissue. The calcium can be in a number of forms, e.g. calcium phosphate, hydroxyapatite, carbonate apatite, amorphous calcium phosphate, etc.

The term “ectopic calcifications” refers to a calcification that may arise in a wide variety of contexts including, but not limited to, calcification of one or more heart valves (e.g. aortic valves), blood vessels, calcifications of lymph nodes, renal calcifications (e.g. nephrocalcinosis), calcifications of muscles and/or tendons, calcifications in the gall bladder, calcifications associated with uremia (e.g. associated with end-stage renal disease), certain cancer growths and/or metastases, calcification associated with blood clot formation, calcification associated with skin pathologies, and the like, either due to physiological alteration or due to other pathologies/drug treatments/aging/environmental causes, or those resulting from genetic diseases associated with gene mutations (ex. Keutel syndrome).

The term “control sample” includes any sample that can be used to establish a base line to be used as reference for future studies or the normal levels in non pathological conditions, and may include tissue or biological fluid samples taken from a healthy mammal or non-mammalian organism or samples mimicking physiological fluid.

The phrase “testing a sample for the presence of GRP” includes testing for the presence of GRP protein as well as testing for the presence of nucleic acid molecules encoding the GRP protein. Methods for detecting proteins and nucleic acids are discussed in greater detail below.

The term “antibody” refers to a protein that specifically binds to another molecule that possesses one or more unique antigenic sites.

The term “antigen” is any molecule having a particular site recognized by an antibody. It is apparent that the category of molecules that may be considered antigens under this definition is broad; essentially any molecule that can bind an antibody qualifies as an antigen, including but not exclusively a protein.

“Peptides” are generally greater than 2 amino acids, greater than 4 amino acids, greater than about 10 amino acids, greater than about 15 amino acids, greater than about 20 amino acids, sometimes up to about 50 amino acids. In some embodiments, peptides are between 5 and 15, between 8 and 15 or between 15 and 30 amino acids in length.

“Specific affinity” is the strongly attractive interaction between specific antigens and their corresponding antibodies. The selective attraction between an antigen and its corresponding antibody occurs between the antigenic site, or epitope, of the antigen and a recognition region in the antibody. As such, it may be possible to modify an antigen by altering its molecular weight, without noticeably altering the epitope, so that the modified antigen maintains a specific affinity for the same antibody as the unmodified antigen. An antibody may similarly be modified without eliminating the specific affinity for its antigen. The high specific affinity between antigens and antibodies is the key to the selective capture and isolation of a specified antigen or antibody using an immunoassay.

The term “marker”, “biological marker” or “biomarker”, as used herein, refers to a measurable or detectable entity can be present in a biological sample. Examples or markers include nucleic acids, proteins, or chemicals that are present in biological samples. One example of a biomarker is the presence of GRP protein in any of the processed forms, the presence of the entire protein or a fragment therein, or the presence of corresponding nucleic acids in a biological sample from an organism source.

A “fusion protein” or “fused protein” or “protein fused” as used herein refers to a composite protein, i.e., a single contiguous amino acid sequence, made up of two (or more) distinct, heterologous polypeptides that are not normally fused together in a single amino acid sequence. Fusion proteins can generally be prepared using well-known methods as for example chemical synthesis or recombinant nucleic acid methods. The resulting product or fusion proteins are not found in nature.

### REFERENCES

Barone L M, Aronow M A, Tassinari M S, Conlon D, Canalis E, Stein G S, Lian J B. Differential effects of warfarin on mRNA levels of developmentally regulated vitamin K dependent proteins, osteocalcin, and matrix GLA protein in vitro. J Cell Physiol. 1994, 160(2):255-64.
Belluoccio D, Wilson R, Thornton D J, Wallis T P, Gorman J J, Bateman J F. Proteomic analysis of mouse growth plate cartilage, Proteomics. 2006, 6(24):6549-53.
Borst P, van de Wetering K, Schlingemann R. Does the absence of ABCC6 (Multidrug Resistance Protein 6) in patients with Pseudoxanthoma elasticum prevent the liver from providing sufficient vitamin K to the periphery? Cell Cycle. 2008, 7(11).
Carrino D A, Onnerfjord P, Sandy J D, Cs-Szabo G, Scott P G, Sorrell J M, Heinegård D, Caplan A I, Age-related changes in the proteoglycans of human skin. Specific cleavage of decorin to yield a major catabolic fragment in adult skin. J Biol. Chem. 2003, 278(19):17566-72.
Carrino D A, Rodriguez J P, Caplan A I Dermatan sulfate proteoglycans from the mineralized matrix of the avian eggshell. Connect Tissue Res. 1997, 36(3):175-93.
Cranenburg E C, Vermeer C, Koos R, Boumans M L, Hackeng T M, Bouwman F G, Kwaijtaal M, Brandenburg V M, Ketteler M, Schurgers L J, The Circulating Inactive Form of Matrix Gla Protein (ucMGP) as a Biomarker for Cardiovascular Calcification. J Vasc Res. 2008, 45(5):427-436.
Davies C A, Jeziorskal M, Freemont A J, Herrick A L, Expression of osteonectin and matrix Gla protein in scleroderma patients with and without calcinosis. Rheumatology. 2006, 45:1349-1355
de Boer-van den Berg M A, Verstijnen C P, Vermeer C., Vitamin K-dependent carboxylase in skin. J Invest Dermatol. 1986, 87(3):377-80
Hermansson M, Sawaji Y, Bolton M, Alexander S, Wallace A, Begum S, Wait R, Saklatvala J. Proteomic analysis of articular cartilage shows increased type II collagen synthesis in osteoarthritis and expression of inhibin betaA (activin A), a regulatory molecule for chondrocytes. J Biol. Chem. 2004, 279(42):43514-21.
Hannigan A, Burchmore R, Wilson J B The optimization of protocols for proteome difference gel electrophoresis (DiGE) analysis of preneoplastic skin. J Proteome Res. 2007, 6(9):3422-32
Howe A M, Webster W S: Warfarin exposure and calcification of the arterial system in the rat. Int J Exp Pathol 2000, 81:51-56
Hur D J, Raymond G V, Kahler S G, Riegert-Johnson D L, Cohen B A, Boyadjiev S A, A novel MGP mutation in a consanguineous family: review of the clinical and molecular characteristics of Keutel syndrome. Am J Med Genet A. 2005, 135(1):36-40
Kulman, J D, Harris, J E, Haldeman, B A, Davie, E W, Primary structure and tissue distribution of two novel proline-rich gamma-carboxyglutamic acid proteins. Proc. Natl. Acad. Sci. USA, 1997, 94: 9058-9062
Kulman, J D, Harris, J E, Xie, L, Davie, E W, Identification of two novel transmembrane gamma-carboxyglutamic acid proteins expressed broadly in fetal and adult tissues. Proc. Natl. Acad. Sci. USA, 2001, 98: 1370-1375
Jie, K S, Gijsbers, B L, and Vermeer, C, A specific colorimetric staining method for gamma-carboxyglutamic acid-containing proteins in polyacrylamide gels, 1995 Anal. Biochem. 224, 163-165.
Laizé, V, Martel, P, Viegas, C S B, Price, P A, Cancela, M L, Evolution of matrix and bone gamma-carboxyglutamic acid proteins in vertebrates J. Biol. Chem. 2005, 280: 26659-26668
Lian J B, Pachman L M, Gundberg C M, Partridge R E, Maryjowski M C, Gamma-carboxyglutamate excretion and calcinosis in juvenile dermatomyositis. Arthritis Rheum. 1982, 25(9):1094-1100.
Lian J B, Boivin G, Patterson-Allen P, Grynpas M, Walzer C, Calcergy and calciphylaxis: timed appearance of gamma-carboxyglutamic acid and osteocalcin in mineral deposits. Calcif Tissue Int. 1983; 35(4-5):555-61
Li Q, Jiang Q, Schurgers L J, Uitto J, Pseudoxanthoma elasticum: reduced gamma-glutamyl carboxylation of matrix gla protein in a mouse model (Abcc6−/−). Biochem Biophys Res Commun. 2007, 364(2):208-13.
Lobo I M, Machado S, Teixeira M, Selores M, Calcinosis cutis: a rare feature of adult dermatomyositis. Dermatol Online J. 2008, 14(1):10.
Luo G, Ducy P, McKee M D, Pinero G J, Loyer E, Behringer R R, Karsenty G, Spontaneous calcification of arteries and cartilage in mice lacking matrix GLA protein, Nature. 1997, 386(6620):78-81
Manfioletti, G, Brancolini, C, Avanzi, G, and Schneider, C The protein encoded by a growth arrest-specific gene (gas6) is a new member of the vitamin K-dependent proteins related to protein S, a negative coregulator in the blood coagulation cascade. Mol. Cell. Biol. 1993, 13, 4976-4985
Munroe P B, Olgunturk R O, Fryns J P, Van Maldergem L, Ziereisen F, Yuksel B, Gardiner R M, Chung E, Mutations in the gene encoding the human matrix Gla protein cause Keutel syndrome. Nat. Genet. 1999, 21(1):142-4.
Oliveri M B, Palermo R, Mautalen C, Hübscher 0, Regression of calcinosis during diltiazem treatment in juvenile dermatomyositis. J. Rheumatol. 1996, 23(12):2152-5.
Pombinho A. R., Laize V., Molha D. M., Marques S. M., Cancela M. L., Development of two bone-derived cell lines from the marine teleost Sparus aurata; evidence for extracellular matrix mineralization and cell-type-specific expression of matrix Gla protein and osteocalcin. Cell Tissue Res. 2004, 315:393-406.
Price P A, Williamson M K, Nguyen T M, Than T N., Serum levels of the fetuin-mineral complex correlate with artery calcification in the rat. J Biol. Chem. 2004, 279(3):1594-600.
Price P A, Otsuka A A, Poser A, Kristaponis J W, and Raman, Characterization of a gamma-carboxyglutamic acid-containing protein from boné, Proc. Natl. Acad. Sci. USA 1976, 73: 1447-1451
Price, P A, Urist, M R, and Otawara, Y, Matrix Gla protein, a new gamma-carboxyglutamic acid-containing protein which is associated with the organic matrix of bone, Biochem. Biophys. Res. Comun. 1983, 117, 765-771
Sambrook, J, Fritsch, E F and Maniatis, T, Molecular cloning: A Laboratory Manual. Cold Spring Harbor Laboratory Press, New York, 1989.
Shearer M J, Vitamin K and vitamin K-dependent proteins, Br J. Haematol. 1990, 75(2):156-62.
Simes D C, Williamson M K, Schaff B J, Gavaia P J, Ingleton P M, Price P A, Cancela M L, Characterization of osteocalcin (BGP) and matrix Gla protein (MGP) fish specific antibodies: validation for immunodetection studies in lower vertebrates. Calcif Tissue Int 2004, 74(2):170-80
Simes D C, Williamson M K, Delgado J B O, Viegas C S B, Price P A, Cancela M L, Purification of Matrix Gla protein (MGP) from a marine teleost fish, Argyrosomus regius: calcified cartilage and not bone as the primary site of MGP accumulation in fish. J. Bone Min. Res. 2003, 18: 244-259
Spronk H M, Soute B A, Schurgers L J, Thijssen H H, De Mey J G, Vermeer C, Tissue-specific utilization of menaquinone-4 results in the prevention of arterial calcification in warfarin-treated rats. J Vasc Res 2003, 40: 531-537,
Surmann-Schmitt, C, Dietz, U, Kireva, T, Adam, N, Park, J, Tagariello, A, Onnerfjord, P, Heinegard, D, Schlötzer-Schrehardt, U, Deutzmann, R, von der Mark, K, and Stock, M, Ucma, a novel secreted cartilage-specific protein with implications in osteogenesis. J. Biol. Chem. 2008, 283, 7082-93
Tagariello A, Luther J, Streiter M, Didt-Koziel., Wuelling M, Surmann-Schmitt, C, Stock M, Adam N, Vortkamp A, and Winterpacht A, Ucma—A novel secreted factor represents a highly specific marker for distal chondrocytes. Matrix Biol. 2008, 27, 3-11
Thijssen H H, Vervoort L M, Schurgers L J, Shearer M J Menadione is a metabolite of oral vitamin K. Br J Nutr 2006, 95:260-266
Vanakker O M, Martin L, Gheduzzi D, Leroy B P, Loeys B L, Guerci V I, Matthys D, Terry S F, Coucke P J, Pasquali-Ronchetti I, De Paepe A, Pseudoxanthoma elasticum-like phenotype with cutis laxa and multiple coagulation factor deficiency represents a separate genetic entity. J Invest Dermatol. 2007, 127(3):581-7.
Van Summeren M J, Spliet W G, van Royen-Kerkhof A, Vermeer C, Lilien M, Kuis W, Schurgers L J, Calcinosis in juvenile dermatomyositis: a possible role for the vitamin K-dependent protein matrix Gla protein. Rheumatology (Oxford). 2008, 47(3):267-71.
Vermeer C, Gamma-carboxyglutamate-containing proteins and the vitamin K-dependent carboxylase. Biochem J. 1990, 266(3):625-36.
Hauschka, P. V., Lian, J. B., and Gallop, P. M. Proc. Natl. Acad. Sci. USA 1975, 72, 3925-3929
Vincourt J B, Lionneton F, Kratassiouk G, Guillemin F, Netter P, Mainard D, Magdalou J. Establishment of a reliable method for direct proteome characterization of human articular cartilage. Mol Cell Proteomics. 2006, 5(10):1984-95.
Zhu A, Sun H, Raymond R M Jr, Furie B C, Furie B, Bronstein M, Kaufman R J, Westrick R, Ginsburg D, Fatal hemorrhage in mice lacking gamma-glutamyl carboxylase. Blood. 2007, 109(12):5270-5.

1. A novel vitamin K-dependent protein rich in gammacarboxyglutamate residues (GRP) comprising between 1 to 14, 1 to 15 or 1 to 16 of gammacarboxyglutamate residues (Gla), in substitution of the glutamic acid residues (Glu), accordingly to the species, providing a GRP fully or partially gammacarboxilated.

2. The protein according to claim 1 having the following specific motifs:
a) a signal peptide including 26-27 amino acids;
b) a propeptide domain containing a gamma-carboxylase recognition site, an AXXF motif and a furin-like cleavage site; and
c) a proven Gla-containing mature protein with 65-74 amino acids and containing between 14 and 16 Gla residues, depending on the species.

3. A method for producing the protein of claims 1 to 2 using recombinant techniques comprising the following steps:
a) constructing a cDNA encoding the GRP coding sequence or a chimeric fusion protein product from a specific mRNA coding for GRP;
b) cloning the corresponding cDNA into expression vectors;
c) transforming host cells; and expressing the cDNA or mRNA in the referred host systems,
d) Purification of the protein of claims 1 to 2.

4. The method of claim 3 wherein a cDNA encoding the GRP coding sequence is from animal origin, including human or other mammalian species, or fish or amphibian, or reptile origin.

5. The method according to claims 3 and 4, wherein expression host systems are bacterial, yeast, animal, insect or plant cells.

6. A method for isolating and purifying the native protein of claims 1 to 2, comprising the following steps:
a) introducing the sample into an extraction solution;
b) applying a demineralization step procedure for extracting GRP from calcified tissues samples (only applicable to mineralized samples);
c) applying a dialysis step;
d) a dissolution in a denaturant solution or a solution with pH in the acidic range (1-7);
e) applying an isolation step;
f) applying a purification step.

7. The method according to claim 6 wherein samples are from animal origin, more specifically mammalian origin including human, or fish or amphibian, or reptile origin and wherein samples are tissues, fluid samples, organs, whole organisms and or cell cultures (cells and/or conditioned medium from cultured cells).

8. The method of previous claims 6 to 7 wherein samples are calcified and/or non-calcified.

9. The method of claims 6 to 8 wherein extraction solution is guanidine 1 to 6 M; urea 1 to 4 M or a combination of both.

10. The method of claims 6 to 9 wherein an isolation step is performed by incubating the extracted solution (only applicable to non-mineralized samples) with hydroxyapatite or other calcium mineral phase or surface followed by a demineralization step procedure.

11. The method of claims 6 to 10 wherein purification is achieved by chromatographic methods, namely RP-HPLC, gel filtration affinity chromatography.

12. An IgG antibody comprising the homologous amino acid sequence against a protein as described in claims 1 to 2 containing at least those amino acid residues that can specifically recognize and bind the epitopes contained or part of a sequence with regions A and/or B accordingly to the sequences identified in Table 1.

13. The hybridomas producing the antibodies of the previous claim.

14. A method for producing the antibodies of claim 12 comprising the following steps:
a) using the hybridoma of claim 13;
b) immunizing an animal with peptides of claim 12 and obtaining the serum or eggs from the immunized animal;
c) immunizing an animal with the protein described in claims 1 to 2 and obtaining the serum or eggs from the immunized animal;
d) immunizing a mammal or a chicken with the protein obtained in claim 6 and obtaining the serum or eggs from the immunized animal;
e) immunizing an animal with the protein obtained in claim 3 and obtaining the serum or eggs from the immunized animal.

15. A biomarker for biological analysis comprising the cDNA and gene sequences (including promoter and 5′ and 3′ flanking regions) for the protein of claims 1 to 2 from animal origin, including human or other mammalian species, or fish, or amphibian, or reptile origin.

16. Species-specific mRNA probes according to claim 15, to detect the protein of claims 1 to 2 by in situ hybridization in samples obtained from tissue samples, biopsies, organs, cells, whole organisms.

17. A method for detecting the mRNA of claim 15 coding for the protein of claims 1 to 2 in specific target tissue samples comprising the following steps:
a) Producing the specific mRNA probe of claim 16;
b) in situ hybridization of those probes with the targeted samples;
c) detection of GRP mRNA levels in samples by molecular techniques.

18. The method according to claim 17, wherein specific mRNA probes are species specific for fishes, amphibians, reptiles, mammals, and human, comprising any region of the cDNA with more than 25 bp of the specific sequence.

19. The method accordingly to claims 17 and 18, wherein tissue samples are biopsies, tissues, organs, cells, whole organisms.

20. A method for detecting and quantifying the mRNA of claim 15, coding for the protein of claims 1 to 2, in specific target tissue including fluid samples, or cell culture samples comprising the following steps:
a) mRNA extraction from the target sample;
b) detection of the mRNA of claim 15 by real-time Time PC or Time PC-ELISA;
c) quantification of the mRNA of claim 15 using specific analysis software.

21. The method of claim 20, wherein tissue samples are biopsies, tissues, organs or whole organisms, wherein fluid sample is blood and wherein cell culture samples are primary or established cell cultures derived from fish, amphibians, reptiles, mammals and human.

22. Use of the methods of claims 17 to 21 to detect and quantify the amount of GRP mRNA, for detecting a disease in a vertebrate selected from the group of coronary atherosclerosis, vascular calcification angiogenesis, or other diseases of the vascular system, diseases affecting bone and cartilage abnormal calcification including joints, diabetes mellitus, ectopic calcification in tumor development, or in skin.

23. A method for detecting and/or quantifying the protein of claims 1 to 2 in samples and comprising the following steps:
a) Obtaining the sample;
b) Incubation of the targeted samples with at least one of the IgG antibodies of claim 13;
c) measuring the amount of the protein of claims 1 to 2 in the samples using molecular techniques.

24. The method according to claim 23 wherein said sample can be selected from a group consisting of blood, serum or plasma sample or other source sample or extract obtained from, for example, biological fluids, cell cultures (from mammalian or non-mammalian origin), biopsies, organs, tissues or even a complete organism.

25. The method according to claims 23 and 24 wherein the amount of the protein of claims 1 to 2 in the sample is measured using an immunoassay, wherein the immunoassay is a competitive immunoassay, an ELISA immunoassay, a radioimmunoassay (RIA), a western-blot based assay, or a dot-blot based assay.

26. The method according to claim 25 wherein the immunoassay utilizes a detectable label selected from the group consisting of radioisotopes, enzymes, fluorescent molecules, chemiluminescent molecules, bioluminescent molecules and colloidal metals to measure the protein.

27. A diagnostic kit for assaying human Gla rich protein in Human serum or plasma samples comprising at least one antibody against an epitope in GRP residues 1-53 or 54-73 or combinations thereof, and the reactants for performing at least one of the methods of claim 25.

28. A method for analysing the presence of GRP polymorphisms and mutations comprising the following steps:
a) Extraction of genomic DNA;
b) amplification by Time PC of specific GRP fragments;
c) sequencing of those amplified fragments;
d) comparison with the wild type sequences of claim 15 for identification of mutations, multiple deletions, insertions, substitutions, and/or SNPs.

29. The method of claim 28, wherein genomic DNA is human DNA from selected normal or diseased populations or from populations thought to be possible targets for presence of mutations in GRP gene based on the observed phenotypes.

30. The method according to claims 28 and 29, wherein genomic DNA is human DNA from fetus, extracted after amniocentesis or from umbilical cord blood.

31. The method according to claims 28 to 30, wherein specific GRP fragments are coding, non-coding and/or regulatory regions (exons, introns, promoters and flanking DNA regions, both 5′ and 3′, respectively).

32. Use of the protein of claims 1 to 2 or a fragment of this protein as a biomarker for a disease in a vertebrate selected from the group of coronary atherosclerosis, vascular calcification angiogenesis, diseases of bone and cartilage including joints, a disease of the vascular system, diabetes mellitus, ectopic calcification in tumor development, or in skin.

33. The use of the method of claims 23 to 26 for monitoring, detecting or determining the risk for calcification of arteries and skin and other soft tissues in a vertebrate, said method comprising: detecting the level of Gla rich protein in samples from said vertebrate, wherein a variation on the level of the GRP as compared to that found in a control, indicates that said vertebrate is at increased risk for calcification of arteries and other soft tissues (or is associated with coronary atherosclerosis, vascular calcification angiogenesis, diseases of bone and cartilage including joints, a disease of the vascular system, diabetes mellitus, or ectopic calcification in tumour development, or in skin, thereby monitoring or detecting coronary atherosclerosis, vascular calcification angiogenesis, diseases of bone and cartilage including joints, a disease of the vascular system, diabetes mellitus, ectopic calcification in the skin or in tumor development.)

34. The method of claim 33 where said vertebrate is more specifically a mammal, including human and wherein said sample is a biopsy or blood.


SEQUENCE LISTING (47 sequences)

SEQ ID NO: 1 - protein, 74 aa, Anolis carolinensis
STKSRDELNAETRQMLAADEQRREYYEEQRNEFENFVEEERDEQDERSREQIEQWREYHYDGLDPPYLYNRHVV

SEQ ID NO: 2 - protein, 74 aa, Ambystoma mexicanum
SPKAQGEINAENRQRLSADEQRREYHEEQRNEFENYVEEEQDEQQERTREQVEQWRQWHYDGLYPSYHYQRHNI

SEQ ID NO: 3 - protein, 74 aa, Acipenser naccarii
STKSKDEVNAENRQRLAADERRREYYEEQRNEFENYVEEERDEQQERNREKTEQWREYHYDGLYPSYQYNRHHI

SEQ ID NO: 4 - protein, 74 aa, Bos taurus
SPRFQDEVNVENRQKLWADELRREYHEEQRNEFENFVEEQNDEQGERSREAVEQWRQWHYDGLYPSYLYNRHHI

SEQ ID NO: 5 - protein, 74 aa, Canis familiaris
SPKSRDEANAENRQKLRADELRREYHEEQGNEFENFVEEQNDEQEERSREAIEQWRQWHYDGLYPSYLYNRHHI

SEQ ID NO: 6 - protein, 74 aa, Callithrix jacchus
SPKSHDEANAENRQKLRADELRREYHEEQRNESENFVEEQNDEQEERSREAVEQWRQWHYDGLYPSYLYSRHHI

SEQ ID NO: 7 - protein, 74 aa, Cavia porcellus
SPKSRGEVNVENTQKLRVDELRREYYEEQRNEFENFVEEQKDEQEERDREAVEQWRQWHYDGLFPSYLYNRHHI

SEQ ID NO: 8 - protein, 71 aa, Dicentrarchus labrax
SPRYYSERQAEQRVRLSANERRREYNEEQRNEFENYVEEERDEQNERSREKNEQVREYHYDGLYPRYHWFH

SEQ ID NO: 9 - protein, 74 aa, Danio rerio
AVKTQDEINAEQRQRLAADERRREYHEEQRNKYENYAEEENDEQDERTREKTEQWREFHYDGLDPSYEYNRHTI

SEQ ID NO: 10 - protein, 74 aa, Danio rerio
SPKTYEEYYAEQRVKMSANERRREHLEEQSNEHENYLEEERDEQYERTRERNEQWREFNYDGQYPQYPHHRQYY

SEQ ID NO: 11 - protein, 74 aa, Equus caballus
SPKSRDEVNAENRQKLRADELRREYHEEQRNEFENFVEEQNDEQEERRREAIEQWRQWHYDGLYPSYLYNRHHI

SEQ ID NO: 12 - protein, 74 aa, Erinaceus europaeus
SPKSQDEVNAENRQKARADELRREYYEEQRNEYENFVEEQNDEQEERSREAIEQWRQWHYDGLYPSYLYNRHHI

SEQ ID NO: 13 - protein, 74 aa, Felis catus
SPKSQDEVNAENRQKLRANELRREYYEEQRNEFENFVEEQNDEQEERSREAIEQWRQWHYDGLHPSYLYNRHHI

SEQ ID NO: 14 - protein, 68 aa, Gasterosteus aculeatus
GAKSQDEINAEQRQILAADERKREFHEQKRNEFESYAEEENDEQDERTRESTEQWREFHYDGMHPPHE

SEQ ID NO: 15 - protein, 74 aa, Homo sapiens
SPKSRDEVNVENRQKLRVDELRREYYEEQRNEFENFVEEQNDEQEERSREAVEQWRQWHYDGLHPSYLYNRHHT

SEQ ID NO: 16 - protein, 74 aa, Ictalurus punctatus
SPYSYTEYVAEHKLQSAASERRREYYEEQSNEYENHLEESRNEQYERNRENAEQWREYHYDGLYPQYPHHRPYV

SEQ ID NO: 17 - protein, 74 aa, Macaca mulatta
SPKSRDEVNAENRQKLRADELRREYYEEQRNEFENFVEEQNDEQEERSREAVEQWRQWHYDGLYPSYLYNRHHI

SEQ ID NO: 18 - protein, 74 aa, Monodelphis domestica
SPKSRDELNAENRQQLRADEHRREYYEEQRNEFENFVEEQNDEQEERSREQIEQWRQWHYDGLYPPYLYNRHRI

SEQ ID NO: 19 - protein, 74 aa, Macropus eugenii
SIKSRDELNAENRQQLRADEHRREYYEEQRNEFENFVEEQNDEQEERSREQIEQWRQWHYDGLYPPYLYNRHRI

SEQ ID NO: 20 - protein, 74 aa, Microcebus murinus
SPPSKDEVNVENRQKLRADELRREHYEEQRNEFENFVEEQNDEQEERSREAVEQWRQWHYDGLYPSYLYNRHHI

SEQ ID NO: 21 - protein, 74 aa, Myotis lucifugus
SPKSRDEVNAENRQKLRADELRREYHEEQRNEFENFVEEQNDEQEERNREAIEQWRQWHYDGLYPSYLYNRHHI

SEQ ID NO: 22 - protein, 74 aa, Mus musculus
SPKSRDEVNAENRQRLRDDELRREYYEEQRNEFENFVEEQRDEQEERTREAVEQWRQWHYDGLYPSYLYNRQNI

SEQ ID NO: 23 - protein, 74 aa, Ornithorhynchus anatinus
SPKSRDEINVETRQQLKADEHRREYFEEQRNEFENFVEEQQDEQDERNREQIEQWRQWHYDGLNPPYQYNRHHV

SEQ ID NO: 24 - protein, 74 aa, Oryctolagus cuniculus
SPKSREEVNAENRQKLRADELRKEYYEEQRNEFENFVEEQNDEQEERSREAVEQWRQWHYDGLYPPYLYNRHHI

SEQ ID NO: 25 - protein, 74 aa, Oryzias latipes
AVKSQDELDAEQRQVIAADERKREFHEEKRNEFESYAEEDNDEVNERSRESTEQWREFHYDGVHPPHEDNRHSN

SEQ ID NO: 26 - protein, 71 aa, Oryzias latipes
SPKYYAELVAEQGMKRAASERRREFNEEQRNEYETYAEEDRDEINERSREMNEQLREYHYDGLYPRFHWFH

SEQ ID NO: 27 - protein, 67 aa, Osmerus mordax
AVKSQDEINAEQRQRLAADERKREYHEEQRNEFEGYAEEEHDEQDERTRESTEQWRQFHYDGMDPSK

SEQ ID NO: 28 - protein, 71 aa, Oncorhynchus mykiss
SAKHEAEVLAEQRVRLSADERRREYYDEQRNEFENYVEEERDEQDERTREKTEQWREFHYDGLYPRYPRGW

SEQ ID NO: 29 - protein, 74 aa, Ochotona princeps
SSKSREEANAENRQKIRADEMRREYYEEQRNEFENFVEEQNDEQEEQRREAVEQWRQWHYDGLYPPYLYNRHHI

SEQ ID NO: 30 - protein, 74 aa, Procavia capensis
SPKSRDEVNVENRQKLRADELRREYYEEQRNEFENFVEEQNDEQEERSREAIEQWRQWHYDGLYPPYLYNRHHI

SEQ ID NO: 31 - protein, 75 aa, Petromyzon marinus
SVKLPRETIAEQTQSLLADKRRTEYREEQIKARENFAEEERSETYEQTREATESWREYHYDGLYPSYRLNRHIPY

SEQ ID NO: 32 - protein, 74 aa, Pongo pygmaeus
SPKSRDEVNAENRQKLRADELRREYYEEQRNEFENFVEEQNDEQEERSREAVEQWRQWHYDGLHPSYLYNRHHI

SEQ ID NO: 33 - protein, 74 aa, Pan troglodytes
SPKSRDEVNAENRQKLRVDELRREYYEEQRNEFENFVEEQNDEQEERSREAVEQWRQWHYDGLHPSYLYNRHHT

SEQ ID NO: 34 - protein, 74 aa, Rattus norvegicus
SPKSRDEVTAENRQKLRDDELRREYYEEQRNEFENFVEEQRDEQEERTREAVEQWRQWHYDGLYPSYLYNRQNI

SEQ ID NO: 35 - protein, 71 aa, Sparus aurata
SPRSYAELQAEQRVKIAANERWREYNEEQRNEHENYAEEARDESDERSRETHEQIREYHYDGLYPRYHWFH

SEQ ID NO: 36 - protein, 71 aa, Salmo salar
SAKHEAEVLAEQRVRLSADERRREYYDEQRDEFENYVEEERDEQDERTREKTEQWREFHYDGLYPRYPRGW

SEQ ID NO: 37 - protein, 74 aa, Sus scrofa
SPRSRDEVNAENRQKLRADELRREYHEEQRNEFENFVEEQNDEQEERSREAVEQWRQWHYDGLYPSHLYNRHHI

SEQ ID NO: 38 - protein, 74 aa, Spermophilus tridecemlineatus
SPKSRDEVNVENRQKLWADELRREYYEEQRNEFENFVEEQKDEQEERRREAVEQWRQWHYDGLYPSYLYNRHRI

SEQ ID NO: 39 - protein, 74 aa, Tupaia belangeri
SPKSRDEVNAESRQKLRADELQREYYEEQRNEFENFVEEQNDEQEERSREAVEQWRQWHYDGLYPSYLYNRHHI

SEQ ID NO: 40 - protein, 74 aa, Tetraodon nigroviridis
GLLKSQDEINAEQRQILAADERRREFHERKRKESESHGEEEPDEEDGRTGESTERWREFHHDGMYPPRGNNRHH

SEQ ID NO: 41 - protein, 71 aa, Tetraodon nigroviridis
SGRYYAELRAEQQVQTSAGRRWRERDEERGSAYETYAEERRDERKEQTRERSEQFREFRSDGLRARQYWFH

SEQ ID NO: 42 - protein, 74 aa, Takifugu rubripes
GLKSQDEINAEQRQVLAADERRREFHEEKRKKFESHAEEEHDEQDERTSESTEQWREFHYDGMYPPHEYNRHHT

SEQ ID NO: 43 - protein, 71 aa, Takifugu rubripes
SGRYHAELLAEQREKIFASERWREHNEKRSNVYENYAEEQRNEQTERSRETSEQIREYHYDGLYPRRYWFH

SEQ ID NO: 44 - protein, 74 aa, Tarsius syrichta
SSKSREEVNAENRQKLRADELRREYHEEQRNEFKNFVEEQNDEQEERIREAVEQWRQWHYDGLYPSYLYNRHHI

SEQ ID NO: 45 - protein, 74 aa, Tursiops truncatus
SSKSREEVNAENRQKLQVDELRKEHYEEQRNEFENFVEEQNDEQEERSREAIEQWRQWHSDGLYPPYLYNRHHI

SEQ ID NO: 46 - protein, 74 aa, Xenopus laevis
STKSQDEINAENRQRLSADERRREYYEEQRNEFENYVEEEQDEQEERSREQIEQWRQWHYDGLSPSYLYQRQNI

SEQ ID NO: 47 - protein, 69 aa, Xenopus tropicalis
SPKSQDEIAERQRLSADERRREYYEEQREFEHVEEEQDEQEERSREQIEQWRQWHYDGLSPSYLYQRQI